(19) United States
(12) Patent Application Publication
(10) Pub. No.: US 2026/0289256 A1
(43) Pub. Date: Sep. 24, 2026

(54) SYSTEM

(71) Applicant: SoftBank Group Corp., Tokyo (JP)

(72) Inventor: Koichi HIROKAWA, Tokyo (JP)

(73) Assignee: SoftBank Group Corp., Tokyo (JP)

(21) Appl. No.: 19/560,301

(22) Filed: Mar. 9, 2026

(30) Foreign Application Priority Data
Mar. 19, 2025 (JP) 2025-044914

(51) Int. Cl. G06N 3/0475; A61B 5/00; A61B 5/0205; A61B 5/021; A61B 5/024; A61B 5/16; G05B 13/02; G06N 3/0455; G06V 10/764; G06V 10/82; G06V 40/16

(52) U.S. Cl. CPC G06N 3/0475; A61B 5/0002; A61B 5/0205; A61B 5/165; A61B 5/486; A61B 5/7203; A61B 5/7267; A61B 5/7275; A61B 5/7278; G05B 13/027; G06N 3/0455; A61B 5/0077; A61B 5/021; A61B 5/024; A61B 2560/0242; A61B 2576/02; G06V 10/764; G06V 10/82; G06V 40/174

(57) ABSTRACT

A system includes a processor that is configured to acquire biometric information by using a sensor, generate a first prompt that instructs a generative AI model to perform an analysis in order to analyze the acquired biometric information, and input the first prompt to the generative AI model, and generate a second prompt for controlling an operation of an information processing apparatus based on an analysis result from the generative AI model, and input the second prompt to the information processing apparatus.

### CROSS-REFERENCE TO RELATED APPLICATION

This application is based on and claims priority under 35 USC 119 from Japanese Patent Application No. 2025-044914 filed on Mar. 19, 2025, the disclosure of which is incorporated by reference herein.

### BACKGROUND

### Technical Field

The present disclosure relates to a system.

### Related Art

Japanese Patent Application Laid-Open (JP-A) No. 2022-180282 discloses a persona chatbot control method executed by at least one processor. The method includes steps of: receiving a user utterance, adding the user utterance to a prompt including a description of a chatbot character and an associated instruction sentence, encoding the prompt, and inputting the encoded prompt to a language model to generate a chatbot utterance responding to the user utterance.

Conventional information processing systems that use biometric information primarily rely on fixed, rule-based logic or narrowly trained models to interpret biometric signals and control device behavior. Such systems often lack flexibility when handling complex or ambiguous biometric patterns, and therefore cannot adequately adapt to diverse users, contexts, or rapidly changing conditions. Furthermore, conventional systems typically do not exploit the generative capabilities of advanced AI models to derive rich, context-sensitive analyses or to generate nuanced support, encouragement, or caring behaviors toward a human user. As a result, these systems have limited ability to provide personalized, emotionally aware assistance or to dynamically control operations of information processing apparatuses based on higher-level understanding of biometric information.

Accordingly, there is a need for a system that can (i) flexibly analyze biometric information by leveraging a generative AI model via prompt-based interaction, (ii) control operations of an information processing apparatus based on analysis results produced by the generative AI model, and (iii) cause the generative AI model to generate actions that provide support, encouragement, or care toward a human user in a natural and adaptive manner.

### SUMMARY

In order to solve the above-described problems, a system according to one aspect of the present invention comprises a processor, wherein the processor is configured to acquire biometric information by using a sensor, generate a first prompt that instructs a generative AI model to perform an analysis in order to analyze the acquired biometric information, and input the first prompt to the generative AI model. The processor is further configured to generate a second prompt for controlling an operation of an information processing apparatus based on an analysis result from the generative AI model, and to input the second prompt to the information processing apparatus. By delegating analytical interpretation of biometric data to the generative AI model via prompt-based instructions, the system can flexibly handle complex biometric patterns and adapt to various users and contexts.

In addition, in another aspect, the processor is configured to acquire the biometric information by using a sensor that measures at least a heart rate and a blood pressure, thereby enabling the system to reliably detect mental and physical states such as stress, fatigue, or relaxation. In a further aspect, the processor is configured to generate a third prompt that instructs the generative AI model to provide information, and to input the third prompt to the generative AI model so as to cause the generative AI model to generate actions for support, encouragement, or care toward a human. Through this configuration, the system can not only control device operations based on biometric analysis but also generate context-sensitive, emotionally supportive behaviors, thereby achieving more personalized and human-centric interaction.

The term “system” refers to an arrangement including at least one processor and one or more related components, devices, or modules that cooperate to perform the functions described in the present specification and claims.

The term “processor” refers to any hardware or combination of hardware and software, such as a CPU, GPU, microcontroller, ASIC, FPGA, or distributed processing circuitry, that is capable of executing instructions to perform the operations described in the present specification and claims.

The term “sensor” refers to any device, module, or circuit configured to detect or measure a physical, physiological, or environmental quantity and to output a corresponding electrical signal or digital data representing that quantity.

The term “biometric information” refers to information relating to a physiological or biological state of a human or other living subject, including, but not limited to, heart rate, blood pressure, skin conductance, body temperature, and any other measurable vital sign or biosignal.

The term “generative AI model” refers to an artificial intelligence model, such as a neural network or large language model, that is configured to generate outputs, including text, control signals, or other data, in response to input information such as prompts.

The term “prompt” refers to data, typically in the form of text or structured information, that is provided as input to a generative AI model in order to instruct the generative AI model to perform a specific operation, such as analysis of biometric information or generation of information or actions.

The term “analysis” refers to processing carried out by the generative AI model in response to a prompt, including interpretation, classification, inference, or extraction of meaning or patterns from the biometric information or other input data.

The term “analysis result” refers to information produced by the generative AI model in response to an analysis prompt, including, for example, an inferred state, a classification, a summary, or any derived data that represents an interpretation of the biometric information.

The term “information processing apparatus” refers to any device or system that performs at least one information processing function, such as a computer, server, client terminal, wearable device, smartphone, home appliance, or control unit, whose operation can be controlled based on prompts or other control information.

The term “operation of an information processing apparatus” refers to any functional behavior or state of the information processing apparatus, including execution of applications, control of user interfaces, start or stop of services, adjustment of outputs such as audio or visual content, or other controllable actions.

The term “support, encouragement, or care” refers to actions or outputs that are intended to assist a human user, to motivate or reassure the user, to express concern for the user's mental or physical condition, or to otherwise provide emotionally or psychologically beneficial interaction.

The term “actions for support, encouragement, or care toward a human” refers to behaviors or outputs generated by the generative AI model, such as spoken messages, displayed messages, recommendations, or control commands to devices, that are designed to support, encourage, or show care for the human user.

### BRIEF DESCRIPTION OF THE DRAWINGS

Exemplary embodiments of the present disclosure will be described in detail based on the following figures, wherein:

FIG. 1 is a schematic diagram illustrating an example of a configuration of a data processing system according to a first exemplary embodiment;

FIG. 2 is a schematic diagram illustrating an example of relevant functions of a data processing device and a smart device according to the first exemplary embodiment;

FIG. 3 is a schematic diagram illustrating an example of a configuration of a data processing system according to a second exemplary embodiment;

FIG. 4 is a schematic diagram illustrating an example of relevant functions of a data processing device and smart glasses according to the second exemplary embodiment;

FIG. 5 is a schematic diagram illustrating an example of a configuration of a data processing system according to a third exemplary embodiment;

FIG. 6 is a schematic diagram illustrating an example of relevant functions of a data processing device and a headset-type terminal according to the third exemplary embodiment;

FIG. 7 is a schematic diagram illustrating an example of a configuration of a data processing system according to a fourth exemplary embodiment;

FIG. 8 is a schematic diagram illustrating an example of relevant functions of a data processing device and a robot according to the fourth exemplary embodiment;

FIG. 9 illustrates an emotion map mapping plural emotions;

FIG. 10 illustrates an emotion map mapping plural emotions;

FIG. 11 is a sequence diagram showing the flow of data processing system processing in Example 1;

FIG. 12 is a sequence diagram showing the flow of data processing system processing in Application Example 1;

FIG. 13 is a sequence diagram showing the flow of data processing system processing in Example 2; and

FIG. 14 is a sequence diagram showing the flow of data processing system processing in Application Example 2.

### DETAILED DESCRIPTION

Description follows regarding an example of exemplary embodiments of a system according to technology disclosed herein, with reference to the appended drawings.

First, explanation follows regarding terminology employed in the following description.

In the following exemplary embodiments, a reference-numeral-appended processor (hereinafter simply referred to as “processor”) may be implemented by a single computation unit, and may be implemented by a combination of plural computation units. The processor may be implemented by a single type of computation unit, or may be implemented by a combination of plural types of computation units. Examples of computation unit include a central processing unit (CPU), a graphics processing unit (GPU), a general-purpose computing on graphics processing units (GPGPU), an accelerated processing unit (APU), and the like.

In the following exemplary embodiments, random access memory (RAM) appended with a reference numeral is memory temporarily stored with information, and is employed as working memory by a processor.

In the following exemplary embodiments, reference-numeral-appended storage is a single or plural non-volatile storage devices for storing various programs and various parameters and the like. Examples of non-volatile storage devices include flash memory (such as a solid state drive (SSD)), a magnetic disk (for example, a hard disk), magnetic tape, and the like.

In the following exemplary embodiments, a reference-numeral-appended communication interface (I/F) is an interface including a communication processor and an antenna or the like. The communication I/F has the role of communicating between plural computers. An example of a communication standard applied for the communication I/F is a wireless communication standard, such as a Fifth Generation Mobile Communication System (5G), Wi-Fi (registered trademark), Bluetooth (registered trademark), and the like.

In the following exemplary embodiments “A and/or B” has the same definition as “at least one out of A or B”. Namely, “A and/or B” may mean A alone, may mean B alone, or may mean a combination of A and B. Moreover, similar logic to “A and/or B” is applied when “and/or” is employed to link three or more items in the present specification.

### First Exemplary Embodiment

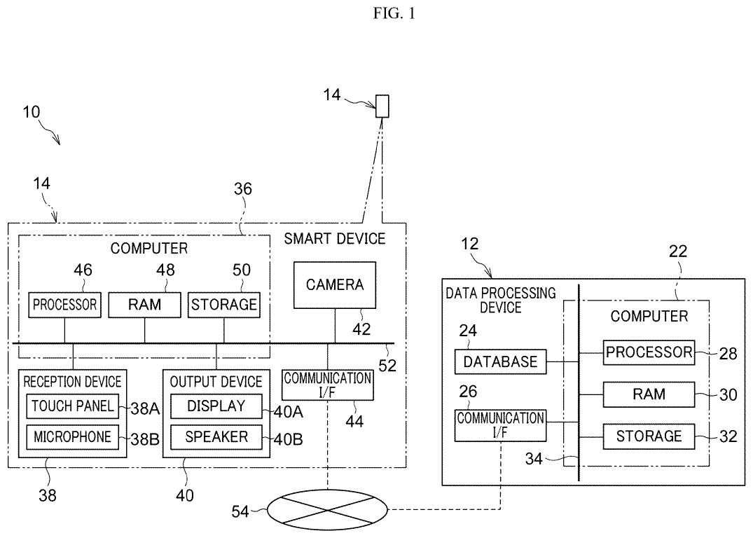

FIG. 1 illustrates an example of a configuration of a data processing system 10 according to a first exemplary embodiment.

As illustrated in FIG. 1, the data processing system 10 includes a data processing device 12 and a smart device 14. A server is an example of the data processing device 12.

The data processing device 12 includes a computer 22, a database 24, and a communication I/F 26. The computer 22 is an example of a “computer” according to technology disclosed herein. The computer 22 includes a processor 28, RAM 30, and storage 32. The processor 28, the RAM 30, and the storage 32 are connected to a bus 34. The database 24 and the communication I/F 26 are also connected to the bus 34. The communication I/F 26 is connected to a network 54. Examples of the network 54 include a Wide Area Network (WAN) and/or a local area network (LAN).

The smart device 14 includes a computer 36, a reception device 38, an output device 40, a camera 42, and a communication I/F 44. The computer 36 includes a processor 46, RAM 48, and storage 50. The processor 46, the RAM 48, and the storage 50 are connected to a bus 52. The reception device 38, the output device 40, the camera 42, and the communication I/F 44 are also connected to the bus 52.

The reception device 38 includes a touch panel 38A, a microphone 38B, and the like for receiving user input. The touch panel 38A receives user input from contact of a pointer (for example, a pen, a finger, or the like) by detecting contact of the pointer. The microphone 38B receives spoken user input by detecting speech of the user. A control unit 46A in the processor 46 transmits data representing the user input received by the touch panel 38A and the microphone 38B to the data processing device 12. A specific processing unit 290 in the data processing device 12 acquires the data indicating the user input.

The output device 40 includes a display 40A, a speaker 40B, and the like for presenting data to a user 20 by outputting the data in an expression format perceivable by the user 20 (for example, audio and/or text). The display 40A displays visual information such as text, images, or the like under instruction from the processor 46. The speaker 40B outputs audio under instruction from the processor 46. The camera 42 is a compact digital camera installed with an optical system such as a lens, an aperture, a shutter, and the like, and with an imaging device such as a complementary metal-oxide semiconductor (CMOS) image sensor or a charge coupled device (CCD) image sensor or the like.

The communication I/F 44 is connected to the network 54. The communication I/F 44 and the communication I/F 26 perform the role of exchanging various information between the processor 46 and the processor 28 over the network 54.

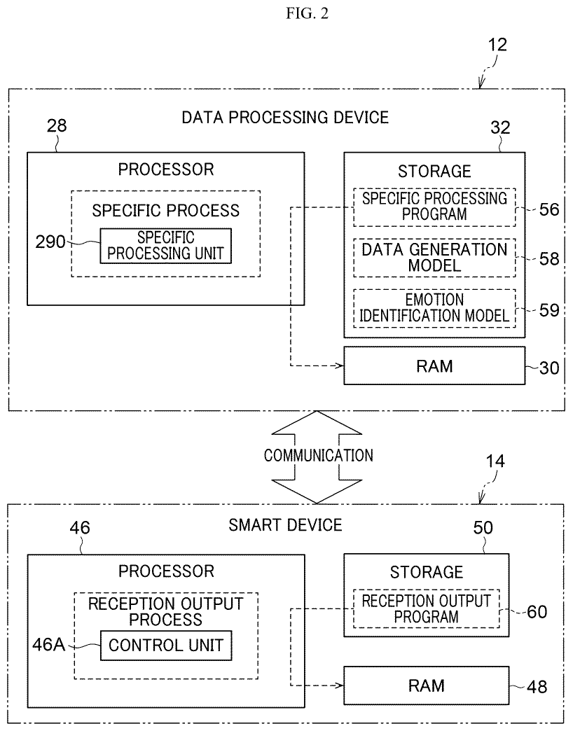

FIG. 2 illustrates an example of relevant functions of the data processing device 12 and the smart device 14.

As illustrated in FIG. 2, specific processing is performed by the processor 28 in the data processing device 12. A specific processing program 56 is stored in the storage 32. The specific processing program 56 is an example of a “program” according to technology disclosed herein. The processor 28 reads the specific processing program 56 from the storage 32, and in the RAM 30 executes the read specific processing program 56. The specific processing is implemented by the processor 28 operating as the specific processing unit 290 according to the specific processing program 56 executed in the RAM 30.

A data generation model 58 and an emotion identification model 59 are stored in the storage 32. The data generation model 58 and the emotion identification model 59 are employed by the specific processing unit 290. The specific processing unit 290 uses the emotion identification model 59 to estimate an emotion of a user, and is able to perform the specific processing using the user emotion. In an emotion estimation function (emotion identification function) that uses the emotion identification model 59, various estimations, predictions, and the like are performed related to emotions of the user, include estimating and predicting the emotion of the user, however, there is no limitation to such examples. Moreover, estimation and prediction of emotion also includes, for example, analyzing (parsing) emotions and the like.

Reception and output processing is performed by the processor 46 in the smart device 14. A reception and output program 60 is stored in the storage 50. The reception and output program 60 is employed by the data processing system 10 in combination with the specific processing program 56. The processor 46 reads the reception and output program 60 from the storage 50, and in the RAM 48 executes the read reception and output program 60. The reception and output processing is implemented by the processor 46 operating as the control unit 46A according to the reception and output program 60 executed in the RAM 48. Note that a configuration may be adopted in which a similar data generation model and emotion identification model to the data generation model 58 and the emotion identification model 59 are included in the smart device 14, and these models are used to perform similar processing to the specific processing unit 290. The reception and output program is implemented by the processor 46 operating as the control unit 46A according to the reception and output program 60 executed in the RAM 48.

Note that devices other than the data processing device 12 may include the data generation model 58. For example, a server device (for example, a generation server) may include the data generation model 58. In such cases, the data processing device 12 performs communication with the server device including the data generation model 58 to obtain a processing result (prediction result or the like) obtained using the data generation model 58. The data processing device 12 may be a server device, and may be a terminal device owned by the user (for example, a mobile phone, a robot, a home electrical appliance, or the like). Next, description follows regarding an example of processing by the data processing system 10 according to the first exemplary embodiment.

### Example 1

Description follows regarding a flow of the specific processing in an Example 1. The units of the system described below are implemented by the data processing device 12 and the smart device 14. The data processing device 12 is called a “server” and the smart device 14 is called a “terminal”.

Conventional computer-implemented systems for monitoring biological information and supporting a human's mental and physical state generally perform fixed rule-based analysis on sensor data and output static messages to a user interface. Such systems suffer from multiple technical limitations.

First, existing architectures typically treat numerical sensor streams and natural language generation as separate, loosely coupled processes. A biological signal processing module may compute simple statistics, and a separate text template engine may insert these statistics into pre-defined message templates. This separation prevents the system from exploiting the full expressive capacity of modern generative AI models, results in low flexibility in adapting output content to diverse physiological patterns, and requires frequent manual updates of templates, thereby increasing maintenance overhead in software and firmware.

Second, conventional systems lack an integrated mechanism to construct and refine machine-interpretable natural language inputs, that is, prompt sentences, for a generative AI model on the basis of time-series features and changes from a reference state of biological information. As a result, the generative AI model is often under-utilized or provided with generic prompts that do not tightly reflect the current computational analysis of the sensor data. This mismatch degrades the quality of the generated analysis and guidance, reduces the relevance of notifications, and limits the overall effectiveness of the system as a computing platform for real-time state estimation.

Third, existing systems generally do not implement a closed-loop feedback architecture that binds user evaluation of notifications back to the generation logic of prompts and the internal parameters of the generative AI model. Although users may indicate whether a notification is useful, such feedback is often stored only as coarse application-level logs and is not systematically associated, at the machine level, with the exact prompt sentence and analysis result that produced the notification. This lack of structured correspondence prevents the computing system from autonomously improving its inference and generation pipelines, requires manual reconfiguration to adjust behavior, and constrains the scalability and robustness of the system in heterogeneous usage environments.

Fourth, from a computer technology standpoint, there is no standardized procedure in many systems for (i) transforming raw time-series biometric measurements into feature sequences suitable for a generative AI model, (ii) combining those feature sequences with dynamically constructed prompt sentences, and (iii) using explicit user feedback as supervised or semi-supervised learning signals to update both the model and the prompt generation process. This absence of a cohesive processing pipeline leads to inefficient use of compute resources, suboptimal memory structures for storing time-series and feedback relationships, and difficulty in tuning model behavior without extensive manual engineering.

Accordingly, there is a need for an improved computer-implemented system and server-side processing architecture that can: (1) systematically acquire and preprocess time-series biological information into feature sequences; (2) generate context-sensitive prompt sentences reflecting change trends and deviations from reference states; (3) jointly supply numerical features and prompt sentences to a generative AI model to obtain analysis results and notification content; and (4) collect, store, and exploit structured user feedback to automatically refine the generative AI model and the prompt generation process. By addressing these issues, the present invention improves the operation of the underlying computer system as a platform for adaptive state estimation and personalized guidance, rather than merely automating manual mental health support tasks.

The specific processing by the specific processing unit 290 of the data processing device 12 in Example 1 is realized by the following means.

The present invention provides a server comprising a processor configured to acquire time-series biological information from a plurality of detection devices, store the acquired biological information as time-series data in a storage device, perform preprocessing computation on the stored biological information and generate, for each predetermined time window, a sequence of feature values, generate, on the basis of the sequence of feature values and a difference between the biological information and a reference state of the biological information, a prompt sentence in natural language that describes a trend of change in the biological information and a mental and physical state of a human, input the prompt sentence and the sequence of feature values as input data to a generative AI model to cause the generative AI model to generate natural language output including an analysis result regarding the mental and physical state of the human and notification content to be presented to the human, transmit, in accordance with the analysis result, a message including the notification content to an information processing apparatus operated by the human, acquire evaluation information from the information processing apparatus, the evaluation information being given by the human in response to the message, and store a correspondence among the evaluation information, the prompt sentence, and the analysis result, and generate, on the basis of the correspondence, learning data for updating the generative AI model or processing for generating the prompt sentence, and improve the generative AI model or the processing for generating the prompt sentence. This enables an integrated computer-implemented pipeline in which time-series biometric signals are converted into feature sequences and context-aware prompt sentences, supplied as structured inputs to a generative AI model, and iteratively refined through explicit user feedback, thereby improving the technical performance of the server in state estimation accuracy, relevance of generated notifications, adaptability of model behavior, and efficiency of model update and prompt generation without requiring manual rule engineering.

The term “biological information” refers to measurable physical or physiological quantities of a living subject, such as heart rate, blood pressure, skin electrical characteristics, body temperature, and other vital signs, acquired as numerical or symbolic data by one or more sensors.

The term “detection device” refers to any hardware device configured to sense and output biological information, including but not limited to body-worn devices, portable information terminals, and sensor modules mounted on such devices.

The term “time-series data” refers to biological information represented as a sequence of data points associated with timestamps or time indices, enabling chronological analysis of changes over time.

The term “storage device” refers to any memory or data retention component, such as a volatile memory, non-volatile memory, or external storage apparatus, configured to store time-series data and related information for subsequent processing.

The term “feature value” refers to a numerical or symbolic value derived from biological information by computation, including raw sensor values, aggregated values, statistical measures, or other transformed quantities used as input to a model.

The term “sequence of feature values” refers to an ordered set of feature values arranged along a time axis or a logical index, representing the evolution of one or more features over a predetermined time window.

The term “reference state of the biological information” refers to a baseline or comparative state of biological information, such as a typical or historical pattern of measurements for a subject or a population, used to determine differences or deviations in current measurements.

The term “trend of change” refers to a pattern in which biological information or feature values vary over time, including increases, decreases, oscillations, and other temporal behaviors discerned from the time-series data.

The term “mental and physical state” refers to an estimated condition of a subject's psychological and physiological status, such as relaxed, stressed, fatigued, or excited, inferred from biological information and related features.

The term “prompt sentence” refers to a natural language expression constructed for input to a generative AI model, the expression describing analysis conditions, contextual information, or instructions that guide the model's generation of output.

The term “generative AI model” refers to a machine learning model configured to generate data, such as natural language text, on the basis of input data including prompt sentences and feature values, and to output analysis results and notifications.

The term “natural language output” refers to text data expressed in a human language, generated by the generative AI model, and including at least an analysis result and notification content for presentation to a human.

The term “analysis result” refers to information indicating an inferred mental and physical state of a subject or related assessment, generated by processing biological information and feature values with the generative AI model.

The term “notification content” refers to information, expressed in natural language, intended to be presented to a subject, including explanations, warnings, suggestions, or action proposals based on the analysis result.

The term “information processing apparatus” refers to any computing apparatus operated by a human, such as a terminal device, portable device, or client system, configured to receive messages, present notifications, and send evaluation information.

The term “message” refers to a data structure transmitted from the server to the information processing apparatus, including at least the notification content and optionally additional metadata for presentation or logging.

The term “evaluation information” refers to data representing a human's reaction or assessment of a message or notification, such as a rating, a binary indication of usefulness, or a textual comment.

The term “correspondence” refers to an association or mapping stored in the storage device between evaluation information and the prompt sentence and analysis result that led to the message evaluated by the human.

The term “learning data” refers to data constructed from the correspondence among evaluation information, prompt sentences, and analysis results, used to adjust parameters of the generative AI model or modify processing for generating prompt sentences.

The term “processing for generating the prompt sentence” refers to computational procedures that determine the content and structure of the prompt sentence, including selection of elements, composition rules, and transformation of feature values and reference state differences into natural language.

In one embodiment, a server, one or more terminals, and a user cooperate to implement the invention.

The server constitutes a data processing apparatus equipped with at least one processor, a main memory, a non-volatile storage device, and a communication interface. The server runs an operating system such as a general-purpose server operating system on hardware including a central processing unit and, optionally, a graphics processing unit. The server uses a database management system such as a relational database, an application framework implemented in a programming language such as a scripting language, and machine learning libraries such as a numerical computation library and a deep learning framework.

The terminal constitutes an information processing apparatus operated by the user. The terminal is realized by a portable information terminal such as a smartphone, or by a body-worn device such as a smartwatch. The terminal includes at least one sensor, such as a heart rate sensor, a blood pressure sensor, a skin electrical resistance sensor, and a temperature sensor. The terminal runs an application program that periodically acquires sensor measurements via an operating system API, associates the measurements with timestamps and a user identifier, and transmits the measurements to the server via a wireless communication link such as a wireless local area network or a mobile communication network.

The user wears or carries the terminal or a separate body-worn device that communicates with the terminal. The user permits the terminal to collect biological information during daily activities. The user further interacts with the terminal by viewing notifications displayed on a screen of the terminal and by inputting evaluation information such as a rating or a comment regarding the usefulness of the notifications.

The server receives the biological information from the terminal through the communication interface. The server stores the received biological information in the storage device as time-series data. The server uses the database management system to maintain tables in which each record includes a user identifier, a timestamp, and at least one feature such as heart rate, blood pressure, skin electrical characteristics, or body temperature. The server indexes the records by user identifier and timestamp so that the server can efficiently retrieve biological information for a given user and a given time range.

The server uses a numerical computation library to perform preprocessing computation on the stored time-series data. The server retrieves, for each user, a recent time interval of biological information such as the last several minutes. The server resamples the data to a fixed sampling interval, performs interpolation when samples are missing, and removes outlier values that deviate from a plausible physiological range. The server aggregates values into time windows, for example by computing a mean, a variance, and a rate of change of heart rate and skin electrical resistance per window. The server thereby generates, for each time window, a sequence of feature values representing the temporal behavior of the biological information.

The server calculates, for each user, a reference state of the biological information. In one embodiment, the server computes the reference state as a long-term average and variability of each biological signal for that user over a training period. In another embodiment, the server uses a population-level model to estimate a baseline appropriate for the user's demographic attributes. The server stores the reference state in the database and updates it as new biological information is accumulated.

The server generates a prompt sentence in natural language based on the sequence of feature values and a difference between the biological information and the reference state. The server uses a prompt generation module implemented in the application framework. The prompt generation module reads the computed feature values and the deviations from the reference state, compares the deviations with predetermined thresholds, and selects templates and phrases accordingly. The server combines numerical values with descriptive phrases to form a prompt sentence that describes a trend of change in the biological information and an inferred mental and physical state.

For example, the server generates a prompt sentence such as:

“The user's average heart rate in the last 5 minutes is 98 beats per minute, which is significantly higher than the user's baseline of 70 beats per minute. The skin electrical resistance has decreased by approximately 25 percent compared to the baseline, and the blood pressure is slightly elevated. Based on these physiological changes, infer the user's mental and physical state and generate a short, empathetic notification message that suggests concrete relaxation techniques suitable for the workplace.”

In another example, the server generates a prompt sentence such as:

“The user's heart rate has increased steadily over the last 10 minutes, while skin electrical resistance has decreased and body temperature remains within the user's normal range.

Determine whether this pattern indicates a stress state and generate a brief message advising safe and discreet relaxation methods in a public environment.”

The server inputs both the prompt sentence and the sequence of feature values as input data to a generative AI model. In one embodiment, the server implements the generative AI model as a neural network including a first encoder for textual input and a second encoder for numerical feature input. The first encoder is a transformer-based language encoder with multiple self-attention layers, and the second encoder is a recurrent neural network or a temporal convolutional network that processes the sequence of feature values. The server concatenates or otherwise fuses internal representations from the two encoders and passes them to a decoder network that generates natural language output token-by-token.

The server trains the generative AI model in advance using supervised learning. The server uses a training dataset that includes historical sequences of biological information, corresponding prompt sentences, and desired natural language outputs that include analysis results and notification content. The server defines a loss function that combines a cross-entropy loss for word prediction in the natural language output with a classification loss for predicted mental and physical state labels. The server updates the weights of the neural network using an optimization algorithm such as stochastic gradient descent or adaptive moment estimation. The server uses data augmentation techniques such as time scaling, noise injection, or random masking of features to improve robustness.

The server executes inference of the trained generative AI model on the hardware accelerators such as the graphics processing unit when available. The server tokenizes the prompt sentence into a sequence of word indices, converts the feature sequence into a numerical tensor, and feeds both streams into the respective encoders. The server then runs the decoder with beam search or greedy decoding to generate the natural language output. Because the feature sequence is processed by a specialized temporal encoder and combined with a contextual prompt sentence, the generative AI model can capture subtle temporal patterns of biological information that conventional template-based systems cannot represent.

The server receives, as output of the generative AI model, an analysis result regarding the mental and physical state of the user and notification content to be presented to the user. The server may also receive an internal probability distribution over possible states and uses this distribution to determine a confidence measure. When the confidence measure exceeds a threshold, the server generates a message that includes the notification content and metadata such as the predicted state and a severity level.

The server transmits the message to the terminal using a push notification mechanism. The server uses a push notification service interface to route the message to a device identifier associated with the terminal. The terminal receives the message via the communication interface, decodes the notification payload, and displays the notification content on a screen. The notification content includes specific guidance for the user, such as: “Let us take a short break. Inhale slowly for four seconds, hold your breath for four seconds, and exhale gently for six seconds. Repeating this for two minutes can help reduce your stress level.” The user views the notification and may follow the suggested actions. The user operates the terminal to provide evaluation information regarding the message. The user may tap a button labeled “Helpful” or “Not helpful,” assign a numerical rating, or enter a free-text comment such as “I still feel anxious even after following the breathing exercise.” The terminal records the evaluation information, associates it with the message identifier and the time of evaluation, and sends the evaluation information to the server via the communication interface.

The server acquires the evaluation information from the terminal and stores a correspondence among the evaluation information, the prompt sentence, and the analysis result that were used to generate the message. The server records this correspondence in a feedback table of the database, in which each entry links a prompt identifier, a representation of the feature sequence, the generated natural language output, and the user's evaluation. The server periodically reads the feedback data and constructs learning data from the correspondence.

The server uses the learning data to update the generative AI model or the processing for generating the prompt sentence. In one embodiment, the server treats evaluation information as an additional supervisory signal. For example, the server assigns higher weights to outputs that received positive evaluations and lower weights to outputs that received negative evaluations. The server then performs fine-tuning of the neural network parameters using the learning data, minimizing a composite loss that penalizes outputs associated with negative evaluations. In another embodiment, the server adjusts thresholds or template selection rules in the prompt generation module based on patterns observed in the feedback. By doing so, the server can selectively emphasize or de-emphasize certain phrases or contextual elements in future prompt sentences.

The server thereby realizes a closed-loop adaptation mechanism that is technically distinct from merely automating human tasks. The server operates on specific data structures (time-stamped sensor records, feature sequences, prompt sentences, and feedback correspondences) and uses a defined neural network architecture and training procedure to improve internal computational behavior. This closed-loop mechanism improves state estimation accuracy because the model uses explicit user feedback to correct misclassifications. It improves processing speed because the server can adapt thresholds and model parameters to reduce unnecessary notifications and to focus computation on cases likely to require intervention. It improves data management because the server maintains structured relations among sensor data, prompts, outputs, and feedback, enabling efficient retrieval and incremental learning.

In another embodiment, the server adjusts the internal behavior of the generative AI model to reduce communication load between the server and the terminal. For example, the server learns, from feedback, that some minor deviations in heart rate do not require a notification for a specific user. The server then changes parameters such as state thresholds in the decision logic so that the server does not send low-importance messages. As a result, the server decreases the number of push notifications and reduces messaging traffic without compromising user support quality. This effect is realized by an internal computation in the server and cannot be easily achieved by manually defined rules.

In still another embodiment, the server uses different neural network architectures for different operation modes. For low-latency operation, the server uses a compact recurrent neural network with fewer layers and reduced parameter count. For high-accuracy operation, the server uses a deeper transformer-based encoder for the feature sequence. The server selects the appropriate architecture based on system load, network latency, or energy constraints, and can dynamically switch between models. This flexible use of model architectures constitutes a technical improvement in computational efficiency and scalability.

The server further provides a function to generate intermediate representations of the feature sequence and prompt sentence that can be cached in memory. For users whose biological information changes slowly, the server reuses cached representations and only updates recent segments, thereby reducing redundant computation. This is a specific technical measure to improve throughput and reduce server-side resource consumption.

The terminal can also implement variations. In one variation, the terminal performs an initial smoothing of sensor data before sending it to the server, using a low-pass filter implemented in the application program. This reduces high-frequency noise and allows the server to process cleaner data, which in turn improves model accuracy and reduces the number of outlier corrections performed at the server. In another variation, the terminal temporarily stores sensor data when the network is unavailable and transmits the buffered data once connectivity is restored, maintaining continuity of the time-series structure without user intervention.

The user may select privacy and sensitivity settings that influence the server's processing. The server stores configuration data indicating which types of biological information should be processed and which kinds of notifications are permitted. The server uses these configuration data to condition the prompt generation and the generative AI model, for example by excluding certain content types or by adjusting the aggressiveness of alerting behavior.

Across these embodiments, the server, the terminal, and the user interact via defined data flows, and the generative AI model and prompt sentence generation are integrated into a concrete computational pipeline operating on structured time-series sensor data. The combination of feature extraction, context-sensitive prompt construction, fused neural network processing, and feedback-driven adaptation produces technical effects such as improved classification accuracy of mental and physical state, reduced false-positive notifications, more efficient use of processing and communication resources, and enhanced robustness to sensor noise and individual variability. These effects arise from specific computer-implemented structures and algorithms and are not merely a computer implementation of a mental health support concept.

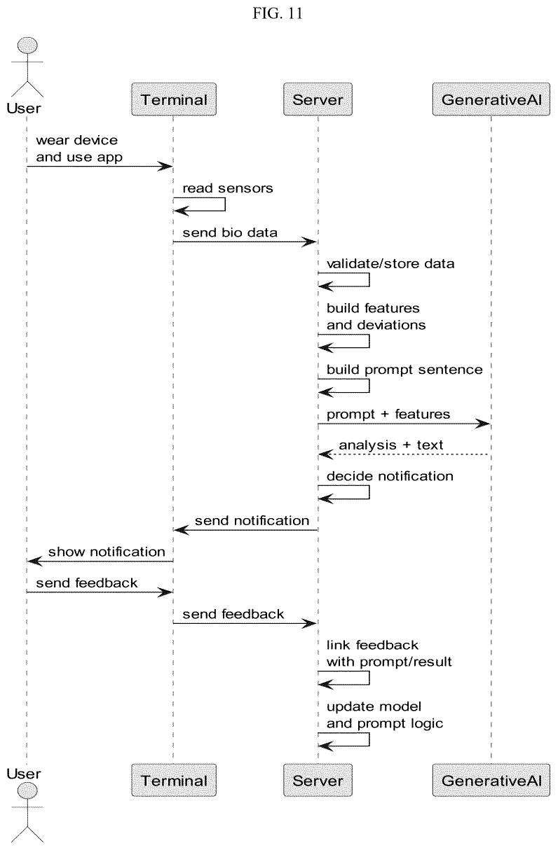

The following describes the processing flow using FIG. 11.

### Step 1:

The terminal acquires biological information.

The terminal uses built-in or connected sensors, such as a heart rate sensor, a blood pressure sensor, a skin electrical resistance sensor, and a temperature sensor, to measure biological information of the user at fixed intervals. The input in this step is a set of raw sensor signals from the hardware sensors. The terminal calls an operating system health API to read current sensor values, associates each value with a timestamp and a user identifier, and forms a measurement record that includes heart rate, blood pressure, skin electrical characteristics, and body temperature. The output of this step is a time-stamped measurement record stored temporarily in the terminal's memory.

### Step 2:

The terminal transmits measurement records to the server.

The terminal periodically aggregates multiple measurement records into a batch for transmission. The input in this step is a collection of locally stored measurement records generated in Step 1. The terminal serializes these records into a structured data format, attaches authentication information, and sends the batch to the server through a secure communication channel such as an encrypted wireless connection. The output of this step is a network message containing the batched biological information delivered to the server.

### Step 3:

The server receives and validates biological information.

The server accepts incoming network messages containing the batched measurement records. The input in this step is the network message transmitted from the terminal in Step 2. The server parses the structured data, verifies authentication tokens, checks that required fields such as user identifier, timestamp, and each biological parameter are present, and validates that each value falls within a plausible physiological range. The server discards or flags invalid records and keeps valid records. The output of this step is a set of validated measurement records ready for storage.

### Step 4:

The server stores biological information as time-series data.

The server writes the validated measurement records into a time-series database or table. The input in this step is the set of validated records from Step 3. The server generates or confirms indices such as user identifier and timestamp, and inserts each record into a storage structure that permits chronological queries. The server may group records by user and by fixed time segments to optimize retrieval. The output of this step is a stored time-series dataset in the server's storage device associated with each user.

### Step 5:

The server retrieves a recent time window of biological information.

The server selects a target user and a specific analysis interval, such as the last several minutes. The input in this step is a query condition including the user identifier and the desired time range. The server executes a query on the time-series dataset and retrieves all records matching the condition, preserving chronological order. The server may limit the number of records to a maximum sequence length. The output of this step is an ordered list of time-stamped biological measurements for the selected time window.

### Step 6:

The server preprocesses the time-series data and generates feature sequences.

The server performs numeric processing on the retrieved measurements. The input in this step is the ordered list of measurements from Step 5. The server resamples the data to a fixed interval by interpolating missing points, removes outliers by applying thresholds or statistical rules, and partitions the time range into smaller sub-windows. For each sub-window, the server computes feature values such as average heart rate, variance of heart rate, average blood pressure, and the rate of change of skin electrical resistance. The server also normalizes each feature using user-specific or global statistics. The output of this step is a sequence of feature vectors, each vector corresponding to one sub-window and collectively representing the temporal pattern of the biological information.

### Step 7:

The server determines a reference state and differences from the reference state.

The server accesses stored baseline information for the user or for a relevant population. The input in this step is the user's feature sequence from Step 6 and the stored reference state values for features such as resting heart rate and typical skin electrical resistance. The server computes differences between current feature values and baseline values, for example by subtracting the baseline from the current mean or by calculating percentage changes. The server may classify each difference as small, moderate, or large using predetermined thresholds. The output of this step is a set of deviation descriptors indicating how each current feature differs from its reference state.

### Step 8:

The server generates a prompt sentence based on feature sequences and deviations.

The server constructs a natural language description to guide a generative AI model. The input in this step is the feature sequence from Step 6 and the deviation descriptors from Step 7. The server selects template fragments that correspond to detected trends such as rising heart rate or decreasing skin resistance, inserts numerical values and qualitative labels, and composes a coherent text. The server ensures that the prompt sentence explicitly requests both an analysis of the user's mental and physical state and a suitable notification message. The output of this step is a prompt sentence in natural language, such as:

“The user's average heart rate in the last 5 minutes is 98 beats per minute, which is significantly higher than the user's baseline of 70 beats per minute. The skin electrical resistance has decreased by approximately 25 percent compared to the baseline, and the blood pressure is slightly elevated. Based on these physiological changes, infer the user's mental and physical state and generate a short, empathetic notification message that suggests concrete relaxation techniques suitable for the workplace.”

### Step 9:

The server prepares combined input data for the generative AI model.

The server converts textual and numerical data into model-ready structures. The input in this step is the prompt sentence from Step 8 and the feature sequence from Step 6. The server tokenizes the prompt sentence into a sequence of word or subword identifiers and embeds these identifiers into continuous vectors using a text embedding layer. The server also arranges the feature sequence into a numerical tensor with dimensions corresponding to time steps and feature channels. The output of this step is a pair of embedded representations: an encoded textual input and a numeric feature tensor, both ready to be processed by the generative AI model.

### Step 10:

The server executes the generative AI model to obtain analysis results and notification content.

The server runs a neural network that processes both the prompt sentence and the feature sequence. The input in this step is the encoded text representation and the numeric feature tensor from Step 9. The server feeds the text representation into a language encoder and the feature tensor into a temporal encoder, then combines the resulting internal states. The server passes the combined state into a decoder network that generates natural language tokens sequentially, and into a classifier layer that outputs a label representing the user's mental and physical state along with confidence scores. The server continues decoding until an end-of-sequence token is produced or a length limit is reached. The output of this step is a natural language output containing an analysis description and notification content, together with structured analysis results such as a state label and a confidence value.

### Step 11:

The server decides whether to send a notification and constructs a message.

The server evaluates the importance of the generated content. The input in this step is the analysis result and the notification content from Step 10, along with decision parameters such as thresholds and user preferences. The server compares the predicted state label and confidence with predetermined criteria, checks recent notification history to avoid excessive alerts, and determines whether a new message should be sent. If a message is warranted, the server formats the notification content into a message structure, adding metadata such as user identifier, severity level, and a message identifier. The output of this step is a finalized message object ready for transmission to the terminal.

### Step 12:

The server transmits the message to the terminal.

The server uses a push notification service or another communication mechanism to deliver the message. The input in this step is the message object created in Step 11 and the device identifier associated with the terminal. The server constructs a network request compatible with the chosen notification service, embeds the message payload, and sends the request through the communication interface. The notification service delivers the payload to the terminal. The output of this step is the arrival of the message at the terminal as a push notification or in-app message.

### Step 13:

The terminal receives and displays the notification content.

The terminal handles the incoming message from the server. The input in this step is the notification payload transmitted in Step 12. The terminal's operating system passes the payload to the application, which parses the contents, extracts the notification text, and displays it on a screen. The terminal may show a banner or open a dedicated view, possibly including additional controls such as buttons for starting a guided exercise. The output of this step is a visual or audible presentation of the notification content to the user.

### Step 14:

The user views the notification and optionally performs suggested actions.

The user reads or listens to the notification displayed by the terminal. The input in this step is the notification content presented in Step 13. The user may follow the suggested behavior, such as performing a breathing exercise or taking a short break, or may ignore the notification. The user's reaction is not necessarily encoded as data in this step, but the user gains awareness of their mental and physical state. The output of this step is a human response, which may include an internal change in condition or a decision to provide explicit feedback.

### Step 15:

The user provides evaluation information through the terminal.

The user interacts with the terminal to indicate their assessment of the notification. The input in this step is the notification interface presented in Step 13 and the user's subjective evaluation formed in Step 14. The user taps a “Helpful” or “Not helpful” button, selects a rating, or enters a free-text comment regarding the notification's relevance or effectiveness. The terminal captures the selected rating and any text, associates them with the message identifier, and stores them temporarily. The output of this step is an evaluation record in the terminal containing structured feedback data from the user.

### Step 16:

The terminal transmits evaluation information to the server.

The terminal prepares the stored evaluation record for transmission. The input in this step is the evaluation record generated in Step 15. The terminal encapsulates the rating, comment, message identifier, and user identifier into a structured data message and sends it to the server through the secure communication channel. The output of this step is a feedback message delivered to the server with explicit evaluation information about a specific notification.

### Step 17:

The server stores correspondences among evaluation information, prompts, and analysis results. The server processes the incoming feedback message from the terminal. The input in this step is the feedback message transmitted in Step 16. The server retrieves the original prompt sentence, feature sequence, and analysis result associated with the message identifier from the database. The server then creates a new record that links the evaluation information to the corresponding prompt sentence and analysis result. The server stores this record in a feedback data structure for later use in model adaptation. The output of this step is an updated feedback dataset containing explicit correspondences among prompts, model outputs, and user evaluations.

### Step 18:

The server generates learning data to update the generative AI model or prompt generation processing.

The server analyzes the accumulated feedback dataset to improve the system. The input in this step is the collection of correspondence records produced in Step 17. The server groups records by prompt pattern, state label, or other attributes, and computes statistics such as average rating or frequency of negative feedback. The server uses these statistics to construct learning samples that emphasize examples with high positive evaluations and de-emphasize or correct examples with negative evaluations. For model updating, the server forms training batches that include the original inputs (prompt sentences and feature sequences) along with target outputs adjusted according to feedback. For prompt generation updating, the server identifies template fragments that correlate with undesired feedback and adjusts selection rules. The output of this step is a set of structured learning data suitable for fine-tuning the generative AI model or for modifying prompt generation rules.

### Step 19:

The server updates internal parameters and processing based on learning data.

The server modifies its computational behavior to reflect the learning data. The input in this step is the learning data created in Step 18 and the current parameters of the generative AI model and prompt generation module. For model adaptation, the server performs fine-tuning by running additional training epochs on the neural network, using learning data and loss functions that penalize outputs associated with negative feedback. For prompt adaptation, the server rewrites configuration parameters, such as thresholds and template selection weights, to increase the likelihood of generating prompts that have historically produced positive evaluations. The output of this step is an updated model and/or updated prompt generation logic that will be used in future executions of Steps 6 through 11, resulting in improved state estimation and notification relevance.

### Application Example 1

Description follows regarding a flow of the specific processing in an Application Example 1. The units of the system described below are implemented by the data processing device 12 and the smart device 14. The data processing device 12 is called a “server” and the smart device 14 is called a “terminal”.

Conventional health-monitoring systems that collect biosignals from human users and display alerts on terminal devices suffer from several technical limitations in terms of data processing efficiency, adaptability, and reliability of automated responses. In many existing architectures, a server performs fixed, rule-based analysis of measurement information such as heart rate and blood pressure, and directly generates static warning messages or control commands for a terminal device. Such rigid pipelines are poorly suited to handle noisy, time-varying biological data, to adapt the system's behavior to different user states, or to scale to large numbers of users with differing conditions.

First, when the server applies only predefined thresholds and static logic to incoming biosignals, the system often generates either excessive false alerts or fails to escalate in a timely manner, thereby degrading the quality of automated decision-making. This results in inefficient utilization of processing resources (for example, repeated, unnecessary notification processing and network transmissions) and a lack of context-sensitive control of terminal-side notification operations. Second, when the generation of warning and explanatory information is hard-coded on the server, updating or personalizing the content requires manual modification of application code and often leads to duplicated logic across different services, making the system difficult to maintain and extend. Third, many systems are not architected to treat prompts to a generative AI model as first-class, dynamically generated control artifacts that are themselves computed from real-time analysis of biosignal streams; instead, generative models, if used at all, are invoked in an ad-hoc manner without structured integration into the core data-processing pipeline. Furthermore, conventional approaches do not adequately address the technical challenge of coordinating multi-stage processing across distributed components (sensors, terminal devices, server devices, and external communication services) while leveraging a generative AI model in a deterministic and auditable way. In particular, there is no systematic mechanism for the server to (i) determine, based on statistical or machine-learning analysis of accumulated measurement information, whether a biological abnormality is present and whether it is sustained, (ii) dynamically construct prompt sentences that encode the current analysis context and desired output behavior of a generative AI model, and (iii) use the resulting AI-generated outputs as structured control information to govern notification and display operations on terminal devices, as well as escalation to pre-registered contacts via communication networks.

As a result, there is a need for an improved computer-implemented system and method that integrates real-time biosignal acquisition, server-side analytical determination of abnormal and sustained abnormal states, and dynamic prompt generation for a generative AI model, in order to provide more flexible, reliable, and efficient control of terminal-side behavior and external notifications. The technical problem to be solved is how to improve the overall operation of the computer system—comprising sensors, terminal devices, server devices, and communication networks—by using dynamically generated prompt sentences and generative AI outputs as structured control signals, thereby enhancing the accuracy, scalability, and adaptability of health-monitoring and alerting functions.

The specific processing by the specific processing unit 290 of the data processing device 12 in Application Example 1 is realized by the following means.

The present invention provides a server comprising a processor and a storage unit, the processor being configured to cooperate with at least one sensor and at least one terminal device to acquire measurement information relating to a biological state of a human subject, to store the measurement information in the storage unit, to analyze the stored measurement information by statistical processing or machine-learning processing in order to determine presence or absence of an abnormal biological state and a duration of the abnormal biological state, to classify the biological state as a sustained abnormal state when the duration meets or exceeds a predetermined time period, to dynamically generate prompt sentences that encode details of the abnormal or sustained abnormal state and desired output behavior of a generative AI model, to input the dynamically generated prompt sentences to the generative AI model so as to cause the generative AI model to generate at least one of an analysis result, a warning message for a user, and a notification message for pre-registered contact information, to generate structured control information including warning information or explanatory information for the user on the basis of outputs obtained from the generative AI model or predefined templates, to transmit the structured control information to the terminal device so as to control a notification operation or a display operation of the terminal device, and, when the sustained abnormal state is determined, to acquire the pre-registered contact information from the storage unit and to transmit notification information based on the notification message to the contact information via a communication network. This enables the computer system as a whole to process biosignal data more accurately and efficiently, to adapt notification and escalation behavior in real time based on server-side analysis, and to leverage the generative AI model as a programmable component whose behavior is controlled by dynamically generated prompt sentences, thereby improving the technical performance, scalability, and reliability of health-monitoring and alert-generation operations.

The term “system” refers to a combination of hardware components and software components including at least one processor, at least one storage unit, at least one sensor, at least one terminal device, and at least one server device that cooperate to perform the functions described herein.

The term “processor” refers to a hardware executing unit, such as a central processing unit or a computing core, that is configured to execute instructions to perform data acquisition, analysis, prompt generation, communication control, and other operations described herein.

The term “storage unit” refers to any memory or recording apparatus, such as volatile memory or non-volatile memory, that is configured to store measurement information, analysis results, contact information, configuration data, prompt sentences, and control information.

The term “sensor” refers to a hardware element or group of hardware elements configured to measure one or more physical or physiological quantities from a human subject, including but not limited to heart rate, blood pressure, and body temperature.

The term “measurement information” refers to digital data representing values obtained by the sensor from a biological state of a human subject, optionally including time information, identification information, and other metadata.

The term “biological state” refers to a physiological condition of a human subject, such as a cardiovascular state, circulatory state, or thermal state, that can be quantitatively represented by measurement information acquired by the sensor.

The term “terminal device” refers to an information processing device used by a user, such as a portable communication device, that is configured to receive control information from a server device and to perform at least one notification operation or display operation based on the control information.

The term “server device” refers to an information processing apparatus, typically located remotely from the terminal device, that includes the processor and the storage unit and is configured to receive measurement information from the terminal device, perform analysis processing, generate prompt sentences, and transmit control information or notification information.

The term “communication network” refers to a wired or wireless data communication infrastructure, such as a packet-switched network, that is configured to transmit information between the sensor, the terminal device, the server device, and external contact destinations.

The term “statistical processing” refers to computational operations that apply statistical techniques, such as averaging, variance computation, threshold comparison, or trend analysis, to measurement information in order to derive analysis results.

The term “machine-learning processing” refers to computational operations that apply at least one algorithmic model trained on data, such as a classification model or regression model, to measurement information in order to infer the presence or absence of specific patterns or states.

The term “abnormal biological state” refers to a biological state determined by statistical processing or machine-learning processing to deviate from a predetermined normal range or expected pattern.

The term “sustained abnormal state” refers to an abnormal biological state that has been determined to continue for at least a predetermined time period based on analysis of measurement information over time.

The term “predetermined time period” refers to a duration condition stored as configuration information or program parameters, which is used as a criterion to decide whether an abnormal biological state is to be treated as a sustained abnormal state.

The term “analysis result” refers to an output of statistical processing or machine-learning processing applied to measurement information, indicating, for example, whether an abnormal biological state is present and, if present, its type and duration.

The term “generative AI model” refers to a computational model that, in response to an input prompt sentence, is configured to generate a sequence of output data, such as natural language text representing analysis results, explanations, or messages.

The term “prompt sentence” refers to a structured input sequence comprising natural language expressions or machine-readable representations that specify to the generative AI model what type of content or behavior is requested.

The term “dynamically generate prompt sentences” refers to the operation of the processor to construct or modify prompt sentences at run-time based on current measurement information, analysis results, stored configuration information, or system state, rather than relying solely on fixed, pre-defined prompts.

The term “warning message” refers to text or other information generated to notify a user that an abnormal biological state has been detected and to advise the user to take one or more actions.

The term “notification message” refers to text or other information generated to inform a contact destination about a sustained abnormal state or related condition of a user, optionally including time information and requested actions.

The term “contact information” refers to data specifying a destination for external communication, such as a telephone number, an electronic mail address, or other addressing information used to send notification information via a communication network.

The term “control information” refers to structured data, generated by the processor, that specifies how a terminal device is to perform a notification operation or a display operation, including contents of messages, presentation style, and timing.

The term “notification operation” refers to an action performed by a terminal device, such as outputting an audio signal, a vibration signal, or a visual alert, in order to draw a user's attention to information.

The term “display operation” refers to an action performed by a terminal device to visually present text, symbols, graphics, or other visual content on a display unit for a user.

The term “user” refers to a human subject whose biological state is monitored by the system and who receives warning information, explanatory information, or other outputs via the terminal device.

The term “explanatory information” refers to content that explains to the user the meaning of an analysis result, an abnormal biological state, or a recommended action, in a natural language or other human-understandable representation.

The term “predefined template message” refers to a message pattern prepared in advance and stored in the storage unit, into which variable elements such as measured values, time information, or user identifiers can be inserted.

The term “structured control information” refers to control information organized in a format such as a data object or record, in which elements such as message text, severity level, and target device operations are represented in a machine-interpretable manner.

The term “psychological burden” refers to a mental stress, anxiety, or discomfort experienced by the user in response to knowledge or suspicion of an abnormal biological state.

The term “dialogue message” refers to text or other content generated for interactive communication with the user, including support, encouragement, or care, which may be presented in a conversational interface.

The term “behavior support” refers to guidance, suggestions, or instructions presented to the user for modifying or selecting actions, such as resting, reducing activity, or seeking assistance, based on the user's biological state.

The term “pre-registered” refers to a state in which information such as contact information or configuration data has been stored in the storage unit before occurrence of a corresponding abnormal biological state or event.

In one embodiment, a server cooperates with a terminal and one or more sensors to monitor a biological state of a user, to analyze measurement information using a combination of statistical processing and machine-learning processing, and to generate prompt sentences for a generative AI model so as to obtain control information that governs notification and display behavior of the terminal and notification behavior toward pre-registered contacts.

A server includes a hardware processor, a main memory, a non-volatile storage device, and a network interface. The hardware processor is, for example, a general-purpose central processing unit or a multicore processor mounted on a server-class computer. The main memory is, for example, a dynamic random-access memory. The non-volatile storage device is, for example, a solid-state drive or a magnetic disk device. The network interface is configured to communicate with a terminal via a packet-switched network using a protocol such as TCP/IP over wired Ethernet or wireless LAN.

A terminal includes a hardware processor, a memory, a display unit, an audio output unit, a vibration actuator, and a wireless communication module. The terminal is, for example, a smartphone, a tablet device, or a wearable information processing device. The terminal executes an application program that acquires measurement information from one or more sensors, transmits the measurement information to the server, and performs notification and display operations according to control information received from the server.

A sensor includes at least one measurement element such as an optical sensor, an electrical sensor, or a pressure sensor. In one specific implementation, a wearable device worn by the user includes an optical sensor that measures a pulse wave to obtain heart rate data, a pressure sensor that estimates blood pressure, and a temperature sensor that measures skin temperature. The wearable device is connected to the terminal via a wireless communication protocol such as Bluetooth Low Energy. Alternatively, the terminal itself includes sensors such as a photoplethysmography sensor, an accelerometer, and a temperature sensor, and the terminal acquires measurement information directly.

The terminal acquires measurement information and associates each measured value with a timestamp and identification information of the user. The terminal maintains the measurement information in a structured format in its memory and transmits the measurement information to the server via the network interface using a secure communication protocol such as HTTPS. The terminal may batch multiple measurement samples into a single data object to reduce communication overhead and to reduce the number of request-response cycles between the terminal and the server. This batching improves communication efficiency and reduces power consumption of the terminal.

The server receives the measurement information and stores it in the storage device in a structured data format. The server uses a data management system, such as a relational database system, to store each sample as a record that includes fields for a user identifier, a timestamp, a heart rate value, a blood pressure value, and a body temperature value. The server maintains indices on at least the user identifier and the timestamp in order to accelerate range queries over time windows. This specific data structure enables efficient retrieval of recent measurement information and reduces processing latency in subsequent analysis.

The server executes statistical processing on the stored measurement information. The server calculates, for each user, a moving average and a moving variance of heart rate and blood pressure over a predefined time window, such as thirty seconds or five minutes, by using vectorized numerical operations in a numerical library. The server compares current values and moving statistics with normal ranges that are stored in a configuration table per user. The server determines that an abnormal biological state is present when at least one measurement value deviates from the corresponding normal range by more than a threshold, or when a deviation persists over consecutive samples. Because the server uses pre-computed moving statistics and indexed retrieval, the server reduces the number of arithmetic operations required for each evaluation and thereby reduces the processing time per user compared to re-computing statistics over entire history.

In another embodiment, the server executes machine-learning processing on the measurement information. The server uses a trained model such as a neural network classifier that receives, as features, a sequence of recent measurement values and derived statistics. For example, the server constructs, as input features, a vector consisting of heart rate samples at fixed time intervals over the last sixty seconds, the differences between consecutive heart rate values, the average heart rate over the interval, and a normalized blood pressure value. The server feeds this feature vector to a trained multi-layer neural network that outputs a probability that the current state is abnormal.

The neural network is, for example, a feed-forward network including an input layer, multiple hidden layers, and an output layer. Each hidden layer applies a linear transformation followed by a nonlinear activation function such as a rectified linear unit. The output layer applies a softmax function to generate probabilities for labels such as “normal”, “mild abnormal”, and “severe abnormal”. The server trains this neural network offline by using a training dataset that contains labeled sequences of measurement information. The server uses a loss function such as cross-entropy loss and an optimization algorithm such as stochastic gradient descent or an adaptive optimizer to update weights. The server regularizes the network using techniques such as dropout and weight decay to improve generalization. The server may perform data augmentation by adding noise within physiological bounds to training samples to increase robustness to measurement noise.

By integrating this trained neural network into the analysis pipeline, the server can detect complex temporal patterns that are not easily captured by simple threshold rules. The server combines the neural network output with statistical threshold checks to improve detection accuracy. For example, the server may require both that a statistical threshold is exceeded and that the neural network indicates a high probability of abnormal state, thereby reducing false positives due to transient sensor errors. This combined approach improves precision and recall of abnormal state detection compared to conventional rule-only systems.

The server determines, based on timestamps of consecutive abnormal determinations, whether the abnormal biological state is a sustained abnormal state. The server maintains, in the storage unit, a table that records time segments during which an abnormal state has been present. The server updates this table each time new analysis results are produced. The server computes the duration of the current abnormal segment by subtracting a start time from a current time, and compares the duration with a predetermined time period such as ten minutes. When the duration exceeds the predetermined time period, the server classifies the state as a sustained abnormal state. This data structure and computation allow the server to make escalation decisions based on temporally aggregated behavior rather than isolated measurements, which leads to more reliable and technically meaningful detection of conditions that require external notification.

The server dynamically generates prompt sentences for a generative AI model based on the analysis results and current measurement context. The server does not merely store fixed prompt strings; instead, the server constructs prompt sentences at run-time by combining template fragments and inserting current values, durations, and classification results. For example, when the server detects a high heart rate that has persisted for forty-five seconds, the server generates a prompt sentence such as:

“The user's heart rate has exceeded the normal range during exercise. Current heart rate: 165 beats per minute. Duration above threshold: 45 seconds. Generate a concise, calm, and clear warning message for the user. The message should advise the user to rest and monitor their condition. Output only the message text in English.”

When the server determines that a sustained abnormal state is present and that an emergency contact should be notified, the server generates a prompt sentence such as:

“The user's heart rate has remained abnormally high for 12 minutes. You are the user's registered emergency contact. Generate a polite but urgent message asking the contact to check on the user. Do not include sensitive medical details, but make clear that assistance may be needed. Output only the SMS text in English.”

The server inputs each prompt sentence to a generative AI model via an application programming interface. The generative AI model is, for example, a transformer-based neural network trained on a large corpus of natural language text. The model includes an encoder-decoder structure with multiple attention layers. The server transmits the prompt sentence as a sequence of tokens and receives, from the model, an output sequence that represents a warning message, an explanatory message, or a notification message.

In another embodiment, the server hosts the generative AI model locally. The server stores model parameters in the storage unit and loads them into memory when the model is used. The server implements the transformer architecture as a sequence of self-attention blocks that compute, for each token, a context-weighted representation using learned query, key, and value matrices. The server generates output tokens by iteratively sampling from a probability distribution predicted by the model at each step, optionally with constraints such as maximum length or end-of-sequence tokens. The server may adjust temperature and top-k sampling parameters to control diversity of generated text.

The server uses outputs of the generative AI model as structured control information rather than as mere human-readable text. The server parses the output, associates it with a severity level according to rules that consider analysis results and keywords present in the generated text, and encapsulates the final message into a data object that is transmitted to the terminal. The server also logs both the prompt sentence and the generated output in the storage unit, which enables later audit and analysis of system behavior. This tight coupling between prompt sentence generation, generative model output, and control information formation contributes to an improvement in the way the computer system orchestrates complex, context-dependent notification behavior without requiring hard-coded branches for every scenario.

The terminal receives the control information from the server. The terminal parses the control information to determine which notification operation and display operation to perform. For example, when the severity level is low, the terminal displays a small popup message at the top of the display unit without producing sound. When the severity level is high, the terminal displays a full-screen alert, plays an audio alarm through the audio output unit, and activates the vibration actuator. The terminal inserts the generated warning message text into the displayed alert. The terminal thereby converts abstract control information into concrete physical outputs that draw the user's attention.

When the server determines that a sustained abnormal state is present, the server retrieves pre-registered contact information from the storage unit, such as a telephone number and an email address. The server uses the generative AI model to generate a notification message for the contact, or alternatively selects a predefined template message, and then uses a communication service or protocol, such as an SMS gateway or an email transfer protocol, to transmit notification information to the contact. In this way, the server not only processes measurement information but also controls external communication equipment indirectly through standardized communication interfaces, which creates a concrete technical effect in the real world by triggering external devices to display or play the notification.

By structuring measurement information, analysis results, prompt sentences, and generated messages into explicit data objects with defined fields, the server can manage and cache intermediate computation results in memory. For example, the server may reuse, across multiple prompt generations, recently computed summary statistics of the user's biological state. This reduces redundant computation and improves the server's throughput. The server may also implement an internal queue that prioritizes prompt generation and AI model invocation for users whose analysis results indicate a higher risk, which optimizes the use of computing resources of the generative AI model and reduces end-to-end latency for high-priority cases. The system provides several technical effects that go beyond mere automation of human decision-making. Because the server integrates indexed storage, efficient statistical computation, and specialized neural network models, the system reduces the time required to determine abnormal and sustained abnormal states compared to manual or naive implementations. The dynamic prompt generation mechanism reduces the amount of static code necessary to cover different combinations of measurement patterns and user contexts, thereby simplifying code maintenance and reducing the risk of inconsistent states. Furthermore, by using a transformer-based generative AI model under the control of explicitly constructed prompt sentences, the server can generate context-appropriate messages that are tailored to current measurement information, which increases the relevance of notifications and reduces unnecessary alerts. This reduction in unnecessary notifications directly translates to lower communication load and lower processing load on both the terminal and the server.

In addition, because the server logs prompt sentences, generated messages, and corresponding measurement information, system developers can analyze these logs offline to refine thresholds, model parameters, and prompt patterns. The server can adjust, for example, the threshold for abnormal heart rate when feedback from users indicates that previous thresholds generated too many false alerts. The architecture thus supports continuous technical improvement of the overall computer system.

In another embodiment, the server generates different types of prompt sentences depending on the target of the message. For user-facing warnings, the server generates prompt sentences that request concise, calm, and clear instructions. For explanatory information, the server generates prompt sentences such as:
“Explain in simple terms what it means when a user's heart rate remains above their safe range for more than 10 minutes. Generate a message that can be shown in a health application, including basic advice on when to seek medical help. Keep the explanation under 150 words.”

For emergency contacts, the server generates prompt sentences for urgent yet polite messages. By explicitly encoding in the prompt sentence the required tone, length, and content constraints, the server exerts fine-grained control over the generative AI model's behavior. This use of prompt sentences as a programmable interface improves the flexibility and extensibility of the system and constitutes a specific improvement in computer-implemented text generation workflows.

In still another embodiment, the server uses different generative AI models or different parameter settings depending on the classification of the abnormal state. For example, the server may use a smaller model or truncated context window for frequent, low-risk warnings to reduce computation time, and a larger, more expressive model for rare, high-risk sustained abnormal states where message nuance is more important. The server manages model selection as part of control information generation, which further optimizes computational efficiency and improves the scalability of the system.

Because the server, terminal, and sensors are configured to operate together according to the above-described structure, the system is able to transform raw biosignal measurements into structured analysis results, and further into context-aware control information and notifications, through a specific sequence of computer-implemented processing steps, data structures, and machine-learning components. This integration produces technical effects such as improved detection accuracy of abnormal states, reduced processing latency, decreased communication overhead, and enhanced robustness of notification behavior, thereby improving the functioning of the computer system itself.

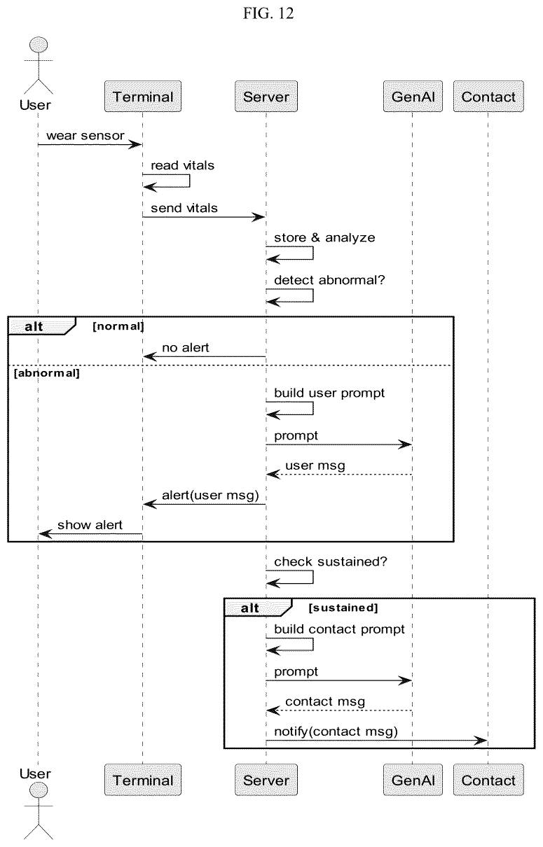

The following describes the processing flow using FIG. 12.

### Step 1:

The terminal acquires measurement information from at least one sensor and prepares the information for transmission.

Input: Raw sensor signals such as optical signals from a pulse sensor, pressure readings from a pressure sensor, and temperature readings from a temperature sensor.

Operation: The terminal converts the raw sensor signals into digital values representing heart rate, blood pressure, and body temperature by using device-specific signal-processing routines. The terminal associates each measured value with a timestamp and a user identifier, and stores the resulting records in a local buffer. The terminal aggregates multiple records into a batch for a defined time window, for example, ten seconds, to reduce communication overhead.

Output: A structured batch of measurement records, each record including at least a user identifier, a timestamp, a heart rate value, a blood pressure value, and a body temperature value.

### Step 2:

The terminal transmits the batch of measurement information to the server via a communication network.

Input: The structured batch of measurement records stored in the local buffer of the terminal.

Operation: The terminal serializes the batch into a data object and establishes a secure communication channel to the server using a protocol such as HTTPS. The terminal attaches authentication information such as a token or certificate and sends the serialized batch to a predefined endpoint on the server. The terminal may compress the batch before transmission to reduce network bandwidth usage.

Output: A network message containing the measurement batch, delivered to the server and ready for reception and processing.

### Step 3:

The server receives the measurement batch and stores the measurement information in a storage unit.

Input: The network message containing the batch of measurement records transmitted by the terminal.

Operation: The server validates the authentication information, parses the received data object, and verifies that required fields such as user identifier and timestamp are present. The server converts each record into a database entry and inserts the entries into a table dedicated to measurement information. The server creates or updates indices on user identifier and timestamp fields to support efficient retrieval.

Output: Persistently stored measurement information in the storage unit, organized as indexed records associated with corresponding users and timestamps.

### Step 4:

The server retrieves recent measurement information for each user and performs statistical analysis to detect abnormal states.

Input: The stored measurement records for a target user within a predetermined time window, such as the past five minutes.

Operation: The server executes a database query that selects records for the target user and orders them by time. The server loads the selected records into memory and computes statistical quantities such as moving averages, moving variances, and maximum and minimum values over sliding sub-windows. The server compares current and aggregated values with normal ranges stored in configuration data. Based on this comparison, the server determines whether the current biological state is normal or abnormal.

Output: An analysis result indicating presence or absence of an abnormal state, and, if abnormal, information such as type of abnormality and magnitude of deviation.

### Step 5:

The server applies machine-learning processing to the measurement information to refine abnormality detection.

Input: The same measurement records retrieved in Step 4 and the analysis result obtained in Step 4.

Operation: The server constructs a feature vector from the measurement records, including recent heart rate samples, changes between consecutive heart rate values, average heart rate, and normalized blood pressure and temperature values. The server inputs this feature vector into a trained neural network classifier stored in memory. The neural network processes the feature vector through its layers and outputs a probability distribution over states such as normal, mild abnormal, and severe abnormal. The server combines this probability distribution with the statistical analysis from Step 4 according to predefined rules, for example, requiring both threshold deviation and high abnormality probability before marking the state as abnormal.

Output: A refined abnormality classification for the user, including a state label and an associated confidence score.

### Step 6:

The server determines whether an abnormal state is sustained based on temporal aggregation of analysis results.

Input: The latest refined abnormality classification from Step 5 and previously stored classifications and timestamps for the same user.

Operation: The server retrieves prior abnormality records for the user from the storage unit and identifies the start time of the current abnormal episode. The server calculates the elapsed time by subtracting the start time from the current time. The server compares the elapsed time with a predetermined threshold duration, such as ten minutes. If the elapsed time meets or exceeds the threshold and the current classification remains abnormal, the server designates the state as a sustained abnormal state.

Output: A determination of whether the user's state is a sustained abnormal state, along with the computed duration of the abnormal episode.

### Step 7:

The server decides whether to generate a warning for the user and to invoke a generative AI model for message generation.

Input: The refined abnormality classification from Step 5, the sustained abnormal state determination from Step 6, and configuration data including notification policies and cooldown intervals.

Operation: The server checks whether the abnormal state satisfies conditions for issuing a user warning, such as magnitude of deviation and absence of recent warnings within a cooldown period. If conditions are met, the server prepares a context summary that includes current measurement values, duration of abnormality, and the identified abnormal type. The server decides whether to generate a message using templates only or by invoking the generative AI model.

Output: A decision to issue a warning or not, and, when issuing a warning, a context summary that will be used to construct a prompt sentence.

### Step 8:

The server constructs a prompt sentence for the generative AI model to generate a warning message for the user.

Input: The context summary prepared in Step 7, including measurement values, duration, and abnormality type, as well as desired message parameters such as tone and length.

Operation: The server selects a base prompt pattern that corresponds to the abnormality type, for example, high heart rate during exercise, and inserts current values into the pattern. The server constructs a complete prompt sentence such as:

“The user's heart rate has exceeded the normal range during exercise. Current heart rate: 165 beats per minute. Duration above threshold: 45 seconds. Generate a concise, calm, and clear warning message for the user. The message should advise the user to rest and monitor their condition. Output only the message text in English.”

The server encodes this prompt sentence in a format suitable for the generative AI model's interface.

Output: A fully formed prompt sentence that encodes the analysis context and the requested behavior of the generative AI model.

### Step 9:

The server inputs the prompt sentence to the generative AI model and obtains a generated warning message.

Input: The prompt sentence constructed in Step 8.

Operation: The server sends the prompt sentence to the generative AI model through an application programming interface, either locally or via a remote service. The generative AI model processes the tokenized prompt sentence through its internal architecture and returns an output sequence representing the warning message. The server receives the output sequence and converts it into a text string. The server may apply post-processing rules to trim unnecessary content, enforce length limits, or correct formatting.

Output: A natural-language warning message text tailored to the current abnormal state and the user's context.

### Step 10:

The server generates control information for the terminal based on the generated warning message and the analysis results.

Input: The warning message text from Step 9, the severity level derived from the analysis results of Steps 4 and 5, and configuration data describing notification behaviors for each severity level. Operation: The server creates a structured control object that includes the warning message text, a severity indicator, and presentation parameters such as whether to use sound, vibration, or full-screen display. The server associates the control object with the user identifier and a timestamp. The server stores a record of this control object for logging purposes and prepares it for transmission to the terminal.

Output: Structured control information specifying how the terminal should notify the user and what message content should be presented.

### Step 11:

The server transmits the control information to the terminal, and the terminal executes notification and display operations.

Input: The structured control information generated in Step 10 and delivered from the server to the terminal through the communication network.

Operation: The server sends the control object to the terminal using a communication mechanism such as a push notification service or a direct network connection. The terminal receives the control object, parses the severity indicator and presentation parameters, and then performs operations such as displaying a message on the display unit, generating sound on the audio output unit, and activating the vibration actuator. The terminal inserts the warning message text into the display area so that the user can read the guidance.

Output: A physical notification experienced by the user in the form of visual, auditory, or tactile signals, along with the displayed warning message.

### Step 12:

The user observes the notification and modifies behavior according to the warning message.

Input: The visual, auditory, or tactile notification produced by the terminal in Step 11, and the warning message content displayed on the terminal's display unit.

Operation: The user reads the warning text, recognizes that an abnormal biological state has been detected, and interprets the recommended action described in the message, such as resting or reducing physical activity. The user may respond by stopping exercise, sitting down, or seeking assistance. The user's behavior change in response to the terminal's operation closes the loop between digital processing and real-world action.

Output: A modified user behavior that is influenced by the server's analysis and the generative AI model's generated message.

### Step 13:

The server determines whether escalation to a pre-registered contact is required based on sustained abnormality.

Input: The sustained abnormal state determination from Step 6, records of previously sent escalation notifications, and escalation policy configuration.

Operation: The server checks if the current sustained abnormal state meets predefined conditions for contacting a pre-registered contact, such as duration exceeding a higher threshold or severity being classified as high. The server ensures that no recent escalation notification has already been sent within a defined cooldown period. If escalation is required, the server prepares a new context summary that includes the duration of the sustained abnormal state and the type of abnormality.

Output: A decision to perform escalation or not, and, when escalation is required, a context summary used for generating a notification message to the contact.

### Step 14:

The server constructs a prompt sentence for the generative AI model to generate a notification message for the pre-registered contact.

Input: The escalation context summary from Step 13, including duration of sustained abnormality and abnormality type, and message requirements such as tone and privacy constraints.

Operation: The server selects a prompt pattern appropriate for contact notifications and inserts the current context into it. The server constructs a prompt sentence such as:

“The user's heart rate has remained abnormally high for 12 minutes. You are the user's registered emergency contact. Generate a polite but urgent message asking the contact to check on the user. Do not include sensitive medical details, but make clear that assistance may be needed. Output only the SMS text in English.”

The server encodes this prompt sentence as input for the generative AI model.

Output: A prompt sentence designed to elicit a suitable notification message for the contact from the generative AI model.

### Step 15:

The server inputs the contact-notification prompt sentence to the generative AI model and generates a contact message.

Input: The prompt sentence produced in Step 14.

Operation: The server transmits the prompt sentence to the generative AI model via the application programming interface. The generative AI model generates a response text that represents the notification message intended for the contact. The server receives the response, converts it into text form, and performs any required post-processing, such as truncating to fit SMS length limitations.

Output: A notification message text suitable for delivery to the pre-registered contact.

### Step 16:

The server sends notification information to the pre-registered contact using external communication services.

Input: The generated contact message text from Step 15 and the contact information, including address or phone number, stored in the storage unit.

Operation: The server composes a communication request that includes the contact's address or phone number and the generated message text. The server sends the request to an external communication service, such as a messaging gateway or email service, using a suitable protocol. The external service delivers the message to the contact's device. The server records the time and content of the notification in the storage unit for logging and auditing.

Output: Delivered notification information to the pre-registered contact, enabling the contact to become aware of the user's sustained abnormal state and to take action if necessary.

It is also possible to incorporate an emotion engine for estimating the user's emotions. That is, the specific processing unit 290 may estimate the user's emotions using an emotion identification model 59, and perform specific processing based on the estimated emotions.

### Example 2

Description follows regarding a flow of the specific processing in an Example 2. The units of the system described below are implemented by the data processing device 12 and the smart device 14. The data processing device 12 is called a “server” and the smart device 14 is called a “terminal”.

Conventional systems that analyze biological information and provide feedback to users typically rely on fixed, rule-based logic or narrowly trained machine learning models. In such systems, a processor generally maps input biological signals, such as heart rate and blood pressure, directly to a limited set of predetermined responses. As a result, these systems suffer from several technical limitations. First, the mapping from sensed data to system behavior is rigid and requires extensive manual tuning when new biological patterns, user contexts, or feedback modalities are introduced. Second, the system behavior is not easily scalable or adaptable, because any change in the feedback strategy necessitates modification of executable code or re-training of specialized models. Third, the interaction between the analysis component and the feedback control component is often fragmented, with no unified mechanism to evolve the end-to-end processing pipeline based on accumulated historical data.

Furthermore, when attempting to incorporate advanced machine learning techniques, conventional architectures typically treat a trained model as a closed component that outputs a single classification result or score. These architectures do not exploit the ability of a generative AI model to flexibly reason over combined inputs such as estimated user state, historical patterns, and available device actions. Consequently, such systems do not efficiently generate or refine high-level control logic for information processing devices, leading to suboptimal or inconsistent use of available output devices such as audio output apparatuses and display output apparatuses. This results in increased development complexity, reduced maintainability, and limited capability to personalize feedback at scale.

There is therefore a need for an improved computer-implemented technique that (i) systematically transforms raw biological information into a structured representation of a user's mental and physical state, (ii) leverages a generative AI model through prompt sentences to dynamically select and refine feedback actions, and (iii) uses stored associations among biological information, estimated states, and executed feedback to iteratively update the prompts themselves. Such a technique should improve the overall operation of the computer system by enabling more flexible, data-driven control of information processing devices while reducing the need for hard-coded rules and manual reconfiguration.

The specific processing by the specific processing unit 290 of the data processing device 12 in Example 2 is realized by the following means.

The present invention provides a server comprising a processor configured to acquire biological information from a detection apparatus, preprocess the acquired biological information, extract feature values based on time-series information, and estimate a mental and physical state of a subject based on the extracted feature values; generate, based on the estimated mental and physical state and supplementary information related to the biological information, an analysis prompt sentence to be input to a generative AI model, and obtain, from the generative AI model, one or more candidate response actions corresponding to the mental and physical state; determine feedback content to be provided to the subject based on the one or more candidate response actions, generate an operation prompt sentence including the determined feedback content, and supply the operation prompt sentence to an information processing device so as to cause the information processing device to control an audio output apparatus or a display output apparatus; and store, in an associated manner, the biological information, the estimated mental and physical state, and the feedback content, and update content of at least one of the analysis prompt sentence and the operation prompt sentence based on the stored information. This enables improved computer functionality in that the server dynamically offloads high-level decision-making to the generative AI model through structured prompt sentences, adaptively refines both analysis and operation prompts according to accumulated data, and thereby achieves more flexible, scalable, and maintainable control of information processing devices for generating user-specific feedback without requiring extensive modification of underlying program code.

The term “biological information” refers to measurable data indicative of a state of a living body, including, for example, heart rate information, blood pressure information, and other physiological parameters acquired by a sensor or detection apparatus.

The term “detection apparatus” refers to a hardware device including at least one sensor configured to measure biological information of a subject and to output the measured biological information in an electrical or digital form.

The term “subject” refers to a living body, such as a human or animal, whose biological information is measured and whose mental and physical state is estimated by the system.

The term “processor” refers to a hardware processing unit, such as a central processing unit or other computing circuitry, configured to execute instructions for acquiring, processing, and using data in accordance with the present invention.

The term “preprocess” refers to performing data processing operations on raw biological information, including, for example, noise reduction, outlier removal, normalization, and temporal aggregation, to generate data suitable for further analysis.

The term “feature values” refers to numerical or categorical values derived from raw biological information, including, for example, statistical quantities, temporal trends, or other extracted parameters used as input to a model or estimation algorithm.

The term “time-series information” refers to biological information that is associated with timestamps or sampling intervals and represents changes in a measured value over time.

The term “mental and physical state” refers to a condition of a subject that includes at least one of a psychological aspect and a physiological aspect, such as a stress level, relaxation level, or other combined state inferred from biological information.

The term “analysis prompt sentence” refers to text data or a structured linguistic representation that is generated based on biological information and an estimated mental and physical state and is input to a generative AI model to request analysis or decision support.

The term “operation prompt sentence” refers to text data or a structured linguistic representation that includes feedback content determined for a subject and is input to an information processing device to cause execution of a specific operation or control behavior.

The term “generative AI model” refers to a machine learning model, such as a language model, configured to output text or other content in response to a prompt sentence, and to generate candidate response actions or other inferential results based on input information.

The term “candidate response actions” refers to one or more proposed actions or behaviors generated by a generative AI model in response to an analysis prompt sentence and intended to address or adapt to the estimated mental and physical state of a subject.

The term “feedback content” refers to information defining an output or action to be provided to a subject, such as playing audio, displaying visual information, or initiating another assistive behavior, which is determined based on candidate response actions.

The term “information processing device” refers to an electronic apparatus, such as a computing device or terminal, configured to receive an operation prompt sentence and to execute control of one or more output devices according to the content of the operation prompt sentence.

The term “audio output apparatus” refers to a hardware device configured to output sound, such as a speaker, earphone, or headset, controllable by the information processing device.

The term “display output apparatus” refers to a hardware device configured to present visual information, such as a display panel, monitor, or head-mounted display, controllable by the information processing device.

The term “terminal device” refers to an end-user computing device, such as a portable information terminal or stationary information terminal, configured to communicate with a detection apparatus and an information processing device over a communication network.

The term “wireless communication” refers to data communication performed without a wired physical connection, including, for example, communication using a short-range or wide-area wireless communication protocol between devices.

The term “communication network” refers to an infrastructure, including wired and/or wireless communication links and network equipment, configured to transmit data between a terminal device, an information processing device, and other components.

The term “supplementary information related to the biological information” refers to information associated with biological information, including, for example, timestamps, device identifiers, environmental conditions, user attributes, or historical measurement context.

The term “assistance actions” refers to executable behaviors or operations intended to support, encourage, or care for a human, including, for example, providing guidance, messages, or content designed to alleviate stress or improve comfort.

In one embodiment, a server, a terminal, and a user cooperate to implement the present invention. The server includes at least one processor, a memory, a non-transitory storage device, and a network interface. The terminal includes a processor, a memory, a wireless communication module, and at least one audio output apparatus and/or display output apparatus. The user wears or otherwise uses a detection apparatus that includes at least one sensor for acquiring biological information such as heart rate and blood pressure.

The server executes a program stored in the memory. The program is implemented, for example, using a general-purpose server operating system and an application stack including an interpreter or runtime environment for a programming language (for example, a scripting language runtime), a numerical computation library (for example, a tensor computation library), and a machine learning framework (for example, a neural network library). The server uses a database management system, such as a relational database management system, to store biological information, extracted feature values, estimated mental and physical states, and feedback content in associated tables.

The terminal executes a client-side program, such as a mobile application or desktop application, which communicates with the detection apparatus via a wireless communication protocol. The terminal receives biological information from the detection apparatus, aggregates the information in a device-local data structure, and transmits the aggregated biological information to the server via a communication network. The terminal also receives operation prompt sentences and feedback content from the server and controls the audio output apparatus and/or display output apparatus accordingly.

The user wears the detection apparatus, such as a wearable monitoring device, around a wrist, arm, chest, or other body part. The detection apparatus includes a sensor module, for example a photoplethysmography sensor for heart rate and a pressure sensing module for blood pressure. The detection apparatus periodically measures heart rate and blood pressure and transmits digital measurement values to the terminal using a short-range wireless communication protocol. The user may also operate the terminal to start or stop monitoring, adjust sensitivity, or provide explicit feedback regarding the effectiveness of the provided feedback content.

The server uses the processor to acquire biological information from the detection apparatus via the terminal. The server stores received data in a database record structure including, for example, a user identifier, a timestamp, a heart rate value, a systolic pressure value, a diastolic pressure value, and device metadata. The server then preprocesses the data using a numerical computation library. The server performs noise reduction by discarding values outside medically plausible ranges, performs interpolation for short gaps, standardizes values to z-scores using user-specific baselines, and aggregates measurements over fixed time windows. The server thereby transforms irregular raw measurements into normalized time-series feature vectors. The server extracts feature values from the preprocessed data, using, for example, a time-window buffer stored in memory. The server computes statistical features such as mean heart rate, variance, maximum, minimum, and slope of heart rate over a preceding interval. The server similarly computes average systolic and diastolic pressures, pulse pressure, and rate of change of blood pressure. The server encodes these features into a fixed-length feature vector, which the server represents as a multidimensional array suitable for input to a neural network model.

The server estimates a mental and physical state of the user using a trained neural network model. In one embodiment, the server uses a feedforward neural network or a recurrent neural network, such as a gated recurrent unit network or a long short-term memory network, implemented using the neural network library. The neural network includes an input layer corresponding to the extracted feature values, one or more hidden layers with nonlinear activation functions, and an output layer that outputs a probability distribution over discrete states (for example, relaxed, normal, stressed) or a continuous stress score. During inference, the server converts the feature vector into a tensor object, performs matrix multiplications and activation functions in the neural network, and obtains the estimated mental and physical state with a numeric confidence value.

The server stores the estimated mental and physical state in the database, associated with the corresponding biological information. The server can also maintain a historical baseline for each user by computing moving averages and variance over longer periods. By comparing current aggregated features to the baseline distribution, the server adjusts the stress evaluation, such as considering a heart rate elevation above 20 percent of the user-specific baseline sustained over a threshold duration as a stressed state.

The server then generates an analysis prompt sentence to be provided to a generative AI model. The server composes the analysis prompt sentence in natural language, embedding the estimated mental and physical state, the key numerical statistics, and a set of candidate assistance actions that the terminal can execute. The server stores a template for the analysis prompt sentence and programmatically fills in parameter values such as current heart rate, baseline heart rate, blood pressure, time of day, and available device capabilities.

For example, the server generates an analysis prompt sentence such as:
“The user's current heart rate has been 20 percent higher than their baseline for the last 10 minutes.
Average heart rate: 110 bpm.
Usual resting heart rate: 80 bpm.
Blood pressure: 140 over 90.
The system has classified the user's state as ‘stressed’.
As a wellness assistant, choose one concrete action to help reduce the user's stress.
   Choose from:
1) Play relaxation music
2) Start a guided breathing exercise
3) Suggest a gentle stretching routine
Return only the number and a short explanation.”

In another example, the server generates an analysis prompt sentence such as:
“Biometric summary:
Heart rate elevation: 25 percent above baseline for 8 minutes.
Blood pressure: moderately elevated.
Context: evening, user at home, audio output available.
Suggest the most suitable type of relaxation music (genre, tempo, and duration) to reduce stress.
Answer in one concise sentence.”
   In a further example, the server uses a simplified format:
“The user is classified as ‘high stress’ based on heart rate and blood pressure trends.
   Available actions:
(A) Play relaxation music
(B) Start a 5-minute breathing exercise
(C) Suggest a short walk

Which action is most appropriate now? Answer with A, B, or C and give one short reason.”

The server sends the analysis prompt sentence to a generative AI model via a network interface. The generative AI model may be hosted on the same server, on another server, or on a remote service accessible through an application programming interface. The server transmits the analysis prompt sentence as part of a request message and receives a response from the generative AI model. The generative AI model implements a large-scale neural network, such as a transformer-based language model, trained on large text corpora and fine-tuned for instruction following behavior. The server uses the generative AI model as a computational engine for synthesizing high-level decision logic from structured prompt sentences, rather than relying on fixed, rule-based mappings.

The server parses the response from the generative AI model to identify one or more candidate response actions. For example, the response may indicate “1) Play relaxation music. Calm instrumental music at a slow tempo (around 60 to 70 beats per minute) for 15 to 20 minutes is recommended.” The server extracts the action identifier (“play relaxation music”) and associated parameters (for example, tempo, duration, music style). The server may apply a post-processing rule set, stored as a configuration in the memory, to normalize or constrain the candidate response actions, such as limiting duration for battery saving or restricting volume at night.

The server determines feedback content to be provided to the user, using both the candidate response actions and local policies. The feedback content is represented as a structured internal record including fields such as action type, media category, content characteristics, duration, and targeted output device. By using structured feedback content, the server can consistently manage and log different types of actions, compare their effectiveness over time, and adapt prompt generation strategies.

The server generates an operation prompt sentence including the determined feedback content. The operation prompt sentence is directed to an information processing device, which may be the terminal itself or another device controlled by the server. The operation prompt sentence is formatted as text instructions describing which device should perform which operation. For example, the server generates an operation prompt sentence such as:
“Play calm plano music at approximately 60 to 70 beats per minute for 15 minutes on the primary speaker at a moderate volume. Start playback immediately and gradually reduce volume during the last 3 minutes.”

In another example, the server generates an operation prompt sentence such as:
“Start a guided breathing exercise on the display. Show a visual circle that expands for 4 seconds and contracts for 4 seconds, and play a soft tone at each transition. Continue for 5 minutes.”

The server transmits the operation prompt sentence to the terminal via the communication network. The terminal receives the operation prompt sentence, parses the text to identify the requested action, and maps the requested action to one or more concrete device operations. The terminal, for example, selects a suitable audio track from a local library or a streaming service, initiates playback through the audio output apparatus using a media playback interface, and configures volume and duration according to the instruction. If the operation prompt sentence describes a guided breathing exercise, the terminal uses its display output apparatus to render animated graphics and optionally play synchronized audio cues.

The server stores the biological information, the estimated mental and physical state, and the executed feedback content in a database, associating them by keys such as user identifier and time interval. The server may also store the corresponding analysis prompt sentence and operation prompt sentence. Over time, the server accumulates a large dataset that maps input biological patterns and context to generated prompt sentences and to outcomes such as user-reported usefulness or indirect measures like subsequent reduction in stress scores.

The server uses these stored associations to update the content of at least one of the analysis prompt sentence and the operation prompt sentence. In one embodiment, the server analyzes historical data offline, using a batch process that computes statistics on which actions most effectively reduce stress levels for particular profiles or time windows. Based on these statistics, the server modifies the prompt templates to bias the generative AI model toward more effective actions, for example by changing the order of candidate actions, refining action descriptions, or adding constraints such as “avoid suggesting stretching when the user is frequently sitting in public transport.” In another embodiment, the server performs online adaptation by tracking short-term effectiveness indicators and dynamically adjusting the instructions embedded in the analysis prompt sentence.

The system thereby improves computer technology in several specific ways. The server restructures raw biological information into time-windowed feature vectors optimized for neural network inference, which increases the accuracy and robustness of state estimation compared to conventional, threshold-based methods. The use of precomputed features and optimized tensor structures reduces the number of required neural network operations and thus reduces latency. The server also reduces communication load by aggregating multiple sensor readings into compact summaries before transmission, rather than streaming every raw sample, thereby decreasing bandwidth usage and network congestion.

The server leverages the generative AI model not as a generic replacement for human reasoning but as a programmable control logic generator driven by prompt sentences. Because the analysis prompt sentence explicitly enumerates available device capabilities and candidate actions, the generative AI model produces decisions that are directly compatible with the actual system configuration. This architecture avoids re-compilation or manual rule updates when new actions or devices are added; instead, the server updates prompt templates and action lists, which the generative AI model uses to generate compatible outputs. This results in improved maintainability and flexibility of the control logic for information processing devices.

The server also employs non-conventional processing sequences and decision rules that go beyond mere automation of human operations. For example, the server updates prompt contents based on algorithmic analysis of multi-dimensional historical data, using clustering or regression analysis to detect which prompt formulations yield more effective responses from the generative AI model. The combination of time-series feature extraction, neural network-based state estimation, and adaptive prompt engineering based on recorded effectiveness constitutes a specific computer-implemented technique designed to optimize both prediction accuracy and control quality. This integrated loop of sensing, modeling, prompting, and adaptation produces technical effects such as reduced error in stress detection, faster convergence to appropriate feedback patterns, and more efficient utilization of network and processing resources.

In alternative embodiments, the server may use different neural network architectures for state estimation, such as a convolutional network applied to time-series windows or a hybrid network that combines a recurrent layer with an attention mechanism. The loss function used during training may be a cross-entropy loss for classification of mental and physical states or a mean squared error loss for regression of a stress score. The server can use gradient-based optimization methods, such as stochastic gradient descent with momentum or adaptive optimization algorithms, for updating model weights during an offline training phase. Data augmentation techniques may be applied to training data, such as adding random noise within physiological bounds or simulating missing segments, to make the model robust to sensor errors and communication interruptions.

In another embodiment, the terminal may implement some or all of the preprocessing and state estimation steps locally, where the terminal uses a compact neural network model optimized for low-power processors. The server then focuses on aggregating results from multiple users and optimizing prompt templates globally. In yet another embodiment, a separate device, such as a media controller, may receive operation prompt sentences directly from the server and control multiple audio and display output apparatuses distributed in an environment.

The user may, in some embodiments, provide explicit feedback regarding the helpfulness of a given action, for instance by selecting “helpful” or “not helpful” on the terminal interface after receiving feedback. The server stores this feedback as additional labels linked to the corresponding biological information, estimated states, and feedback content. The server may use these labels to further refine state estimation models and prompt templates, thereby closing a feedback loop that continuously improves the system's technical performance, including accuracy of state prediction and appropriateness of selected feedback actions.

By grounding the use of a generative AI model in specific data structures, algorithmic flows, and hardware control operations, and by adaptively modifying prompt sentences based on computational analysis of stored associations, the system avoids being a mere abstract mental process. Instead, the system improves the functioning of the computer network and information processing devices themselves by enabling faster, more accurate, and more resource-efficient processing of biological information and control of output apparatuses, leading to concrete technical effects in real-world device operation.

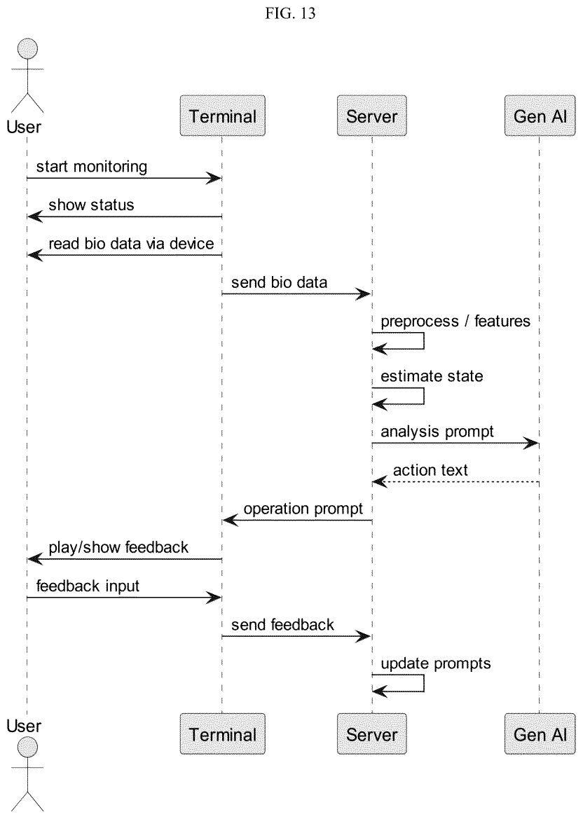

The following describes the processing flow using FIG. 13.

### Step 1:

The user wears the detection apparatus and starts measurement on the terminal.

The user attaches the detection apparatus to the body so that a sensor can measure biological information, such as heart rate and blood pressure, and the user operates an application on the terminal to start monitoring.

Input: the user's physical action of attaching the detection apparatus and starting monitoring on the terminal.

Output: a continuous stream of raw biological measurement values generated by the detection apparatus.

The user ensures that the detection apparatus is properly positioned, that the sensor surface contacts the skin, and that the terminal indicates that monitoring is active.

### Step 2:

The terminal receives biological information from the detection apparatus via wireless communication.

The terminal uses a wireless communication module to establish a connection to the detection apparatus and to receive packets including timestamps, heart rate values, and blood pressure values at predetermined intervals.

Input: raw sensor packets transmitted by the detection apparatus.

Output: a structured sequence of biological records stored in the terminal's memory.

The terminal decodes the received packets, converts binary payloads into numerical values, associates each measurement with a timestamp and user identifier, and appends each record to a local data buffer.

### Step 3:

The terminal aggregates biological information and transmits it to the server.

The terminal groups multiple biological records collected over a defined period into a batch and prepares a transmission message including user identification, measurement values, and device metadata, then sends this message to the server over a communication network.

Input: locally buffered biological records stored in the terminal.

Output: a network message containing an aggregated batch of biological information delivered to the server.

The terminal compresses the batch if necessary, attaches authentication information, and sends the batch using a secure communication protocol to reduce communication overhead and maintain data integrity.

### Step 4:

The server receives and stores raw biological information.

The server accepts the network message from the terminal, verifies authentication, parses the message, and registers each measurement as a record in a database table.

Input: a network message containing aggregated biological information.

Output: database entries representing raw biological information associated with user identifiers and timestamps.

The server opens a database transaction, inserts multiple rows corresponding to each measurement in the batch, and commits the transaction to ensure consistent and durable storage.

### Step 5:

The server preprocesses the biological information and builds time-series segments.

The server loads recent biological records for a particular user from the database, applies noise filtering, removes outliers, fills short gaps, and organizes the data into time-windowed segments.

Input: raw biological information records retrieved from the database.

Output: cleaned and normalized time-series segments ready for feature extraction.

The server examines each record to determine whether the values are within physiologically plausible ranges, discards erroneous values, interpolates missing measurements, and normalizes values relative to user-specific baselines to create stable time windows.

### Step 6:

The server extracts feature values from the preprocessed time-series segments.

The server performs numerical calculations on each time-windowed segment to compute statistical and temporal features, such as mean, variance, maximum, minimum, and slope for heart rate and blood pressure.

Input: cleaned and normalized time-series segments.

Output: fixed-length feature vectors representing the biological state over each time window.

The server iterates over each segment, calculates statistical measures, encodes the resulting values into a predefined vector structure, and stores the feature vectors in memory as arrays suitable for input to a neural network.

### Step 7:

The server estimates the user's mental and physical state using a neural network model.

The server inputs the feature vectors into a trained neural network model and executes inference to obtain probabilities or scores corresponding to mental and physical states, such as relaxed, normal, or stressed.

Input: fixed-length feature vectors derived from biological information.

Output: estimated mental and physical state values, including a state label and a numeric confidence or stress score.

The server converts each feature vector into a model-compatible tensor, feeds the tensor through multiple layers of the neural network, applies activation functions and normalization, and reads the resulting outputs as a predicted state and associated metrics.

### Step 8:

The server updates and maintains a user-specific baseline.

The server compares the current feature values and state estimates with historical data for the same user, computes moving averages and variability, and adjusts the definition of normal ranges accordingly.

Input: current feature vectors and estimated states, and historical records stored in the database.

Output: updated baseline parameters and adjusted criteria for determining deviations such as elevated stress.

The server calculates trends over longer periods, updates baseline statistics in a dedicated table, and stores new baseline parameters that will be used in subsequent evaluations to reduce false positives and improve detection precision.

### Step 9:

The server determines whether the user is in a stressed state based on thresholds and baseline.

The server evaluates whether the current measurements meet specific criteria, such as heart rate being at least a certain percentage above baseline for a minimum duration and blood pressure exceeding defined thresholds, and classifies the current state accordingly.

Input: current feature vectors, estimated mental and physical state, and updated baseline parameters.

Output: a refined classification indicating whether the user is stressed and the degree of stress.

The server applies rule-based conditions combining neural network outputs and baseline comparisons, tags the state as “stressed” when multiple conditions are satisfied, and records this classification as part of the state data for the current time window.

### Step 10:

The server generates an analysis prompt sentence for a generative AI model.

The server constructs a natural-language text that summarizes the biological information, the estimated mental and physical state, and a set of candidate assistance actions that can be executed by the terminal or other devices.

Input: the refined classification of the user's state, numerical statistics of recent biological information, and a list of available assistance actions.

Output: an analysis prompt sentence formatted as a text string.

The server fills a prompt template with parameters such as average heart rate, baseline heart rate, blood pressure, duration of elevation, time of day, and available feedback modalities, and organizes this information into a coherent request to the generative AI model.

### Step 11:

The server transmits the analysis prompt sentence to the generative AI model and receives a response.

The server sends the prompt sentence through a network interface to a generative AI model implemented as a large-scale language model and waits for a response describing one or more candidate response actions.

Input: the analysis prompt sentence prepared by the server.

Output: a response text from the generative AI model specifying a recommended action and optionally associated parameters.

The server encapsulates the prompt in a request message, transmits the message over a communication channel, receives the response text, and stores the request-response pair in memory for later analysis.

### Step 12:

The server parses the response text and identifies candidate response actions.

The server analyzes the response text, locates the indication of a chosen action (for example, a numbered item or action label), and extracts any descriptive parameters such as duration, volume level, or type of content.

Input: response text produced by the generative AI model.

Output: a structured representation of candidate response actions and associated parameters.

The server uses text parsing rules or pattern matching to detect action codes, maps the identified action to an internal action type, and records parameter values in a structured data object representing the candidate response.

### Step 13:

The server determines final feedback content based on candidate response actions and system policies.

The server evaluates the candidate response actions in view of system constraints, user preferences, and safety policies, and selects or adjusts an action to form final feedback content to be provided to the user.

Input: structured candidate response actions, system policies, and user-specific settings.

Output: finalized feedback content, including action type and detailed execution parameters.

The server checks whether the suggested action is permitted at the current time, verifies that required devices are available, may modify duration or intensity, and then commits to a final configuration describing exactly what feedback will be delivered.

### Step 14:

The server generates an operation prompt sentence directed to the information processing device.

The server creates a text instruction that encodes the finalized feedback content in a form that the terminal or another information processing device can interpret and convert into concrete device operations.

Input: finalized feedback content including action type, media characteristics, and target output device.

Output: an operation prompt sentence containing specific instructions for controlling output apparatuses.

The server formats the operation prompt sentence to include directives such as what kind of audio to play, for how long, on which device, and at what intensity, and stores the sentence temporarily in memory prior to transmission.

### Step 15:

The server transmits the operation prompt sentence and feedback parameters to the terminal.

The server sends a control message over the communication network to the terminal, including the operation prompt sentence and any structured parameters needed for execution.

Input: the operation prompt sentence and feedback parameter data.

Output: a network message received by the terminal that instructs the terminal to perform specific output operations.

The server packages the operation prompt sentence in a protocol message, addresses it to the terminal associated with the user, and transmits it using a secure and reliable communication channel.

### Step 16:

The terminal interprets the operation prompt sentence and maps it to device commands.

The terminal parses the received operation prompt sentence, identifies the requested type of feedback, and converts the instructions into API calls or commands to local audio output and display output apparatuses.

Input: the operation prompt sentence received from the server.

Output: a sequence of device-specific commands prepared for execution on the terminal.

The terminal analyzes text segments indicating action type, content type, duration, and volume, selects appropriate media files or streaming sources, and generates corresponding commands for the operating system's media and display subsystems.

### Step 17:

The terminal controls the audio output apparatus and/or display output apparatus to provide feedback to the user.

The terminal executes the generated device commands, causing speakers to play relaxation audio or displays to show visual guidance in accordance with the parameters specified in the operation prompt sentence.

Input: device-specific commands derived from the operation prompt sentence.

Output: actual sensory feedback delivered to the user via audio and/or visual channels.

The terminal starts media playback, maintains playback for the instructed duration, adjusts volume or brightness as needed, and may show status information or guidance messages on the screen while the feedback is being provided.

### Step 18:

The user perceives the feedback and may provide evaluation or control input.

The user listens to the audio output or views the visual output, and optionally uses the terminal interface to indicate whether the feedback was helpful or to adjust or stop the feedback.

Input: sensory feedback from the terminal and user control options presented on the terminal.

Output: user responses such as button presses, ratings, or control commands.

The user may tap buttons such as “Stop,” “Too loud,” or “Helpful,” and the terminal captures these events as input data representing explicit feedback from the user.

### Step 19:

The terminal transmits user feedback and control results back to the server.

The terminal packages user responses, such as effectiveness ratings and control adjustments, along with contextual information, and sends this data to the server for storage and analysis.

Input: user interaction events and contextual data collected on the terminal.

Output: a feedback message delivered to the server describing user reactions to the provided feedback content.

The terminal formats the feedback information as a structured message, attaches timestamps and identifiers, and transmits the message over the established communication network to the server.

### Step 20:

The server records associations among biological information, estimated state, and feedback results and updates prompt strategies.

The server stores user feedback together with the biological information, estimated mental and physical states, generated prompt sentences, and executed feedback content, and uses this stored data to adjust prompt templates or decision policies for future interactions.

Input: user feedback messages, historical biological information, state estimates, analysis prompt sentences, operation prompt sentences, and feedback content records.

Output: updated prompt templates, refined thresholds, and adapted decision parameters that influence subsequent analysis prompt sentences and operation prompt sentences.

The server performs statistical analysis or model-based evaluation on the accumulated data, identifies which types of prompt wording and feedback actions correlate with improved outcomes, modifies prompt templates, and updates configuration parameters so that future processing can achieve higher accuracy and more effective feedback delivery.

### Application Example 2

Description follows regarding a flow of the specific processing in an Application Example 2.

The units of the system described below are implemented by the data processing device 12 and the smart device 14. The data processing device 12 is called a “server” and the smart device 14 is called a “terminal”.

Conventional computer-implemented support systems that use biological information and user input typically rely on fixed rule sets or static models to determine how to process sensor data, how to interpret user state, and how to control downstream devices or present messages. In such systems, logic for interpretation and action selection is hard-coded in the application program, which results in several technical limitations in the operation of the information processing apparatus.

First, when heterogeneous inputs such as physiological signals, behavior information, and emotion information are combined, existing systems often perform separate processing pipelines and then manually merge results. This increases processing latency and computational overhead, and makes it difficult for the processor to adaptively change behavior at runtime. The processor must execute multiple inference modules, each with different interfaces and data formats, which leads to inefficient utilization of memory and processing resources.

Second, in many architectures, natural language generation or use of a generative AI model is treated as a peripheral “add-on” for user interaction, rather than an integral part of the core control logic. As a consequence, the generative AI model is not used to dynamically synthesize or refine machine-readable control instructions. The processor instead must implement intermediate translation layers from fixed internal representations to control commands, which are brittle, difficult to maintain, and not easily extensible to new sensors or new controlled devices.

Third, conventional systems generally do not represent analysis requests and control instructions themselves as structured natural language prompt sentences that are iteratively updated based on continuous sensing and user feedback. The absence of such prompt-centric orchestration restricts the system from leveraging the generative AI model as a flexible reasoning engine that can fuse context, history, and multi-modal state into unified directives. As a result, real-time optimization of both the input to the generative AI model and the control of the information processing apparatus is limited, degrading responsiveness and personalization.

Fourth, known systems frequently require separate components for state estimation, decision logic, and message generation, with each component using different configuration schemes. This fragmented architecture causes redundant computations, repeated data conversions, and complex interface code, which in turn increases the probability of inconsistency between the estimated user state and the actual actions taken by environment control apparatuses or information presentation apparatuses.

Accordingly, there is a need for an improved computer-implemented system and server-side processing technique in which a processor systematically generates, updates, and applies natural-language prompt sentences as first-class control artifacts. Such a system should use generative AI models not only to analyze multi-modal input data but also to derive, in a unified manner, both high-level analysis results and machine-actionable control instructions, thereby improving the efficiency, adaptability, and technical performance of the information processing apparatus as a whole.

The specific processing by the specific processing unit 290 of the data processing device 12 in Application Example 2 is realized by the following means.

The present invention provides a server comprising a processor configured to acquire biological information of a subject by using a sensor apparatus, to generate a first prompt sentence expressed in natural language based on the biological information and at least one of behavior information and emotion information of the subject, to input the first prompt sentence to a generative AI model to obtain, from the generative AI model, an analysis result or an action candidate relating to a physical and mental state or an emotional state of the subject, to generate a control prompt sentence expressed in natural language and specifying an operation content of an information processing apparatus including at least one of an environment control apparatus and an information presentation apparatus based on the analysis result or the action candidate, to input the control prompt sentence to the information processing apparatus to control an operation of the information processing apparatus, to generate a support message or a proposal content using a natural language response obtained from the generative AI model based on the analysis result or the action candidate and at least one of attribute information and history information of the subject, to output the support message or the proposal content to the subject or to an operator corresponding to the subject via a presentation apparatus including at least one of a display apparatus and an audio output apparatus, and to update at least one component of the first prompt sentence and the control prompt sentence based on biological information continuously acquired from the sensor apparatus and response information obtained from the subject, and to iteratively optimize both input to the generative AI model and control of the operation of the information processing apparatus. This enables the server to treat natural-language prompt sentences as machine-interpretable control artifacts, so that the generative AI model can be used as a unified reasoning core for analysis and action selection, thereby improving computational efficiency, reducing interface complexity, and enhancing real-time adaptability of the information processing apparatus.

The term “processor” refers to a hardware and/or software processing unit, such as a central processing unit or a processing circuit, that executes instructions to perform data acquisition, prompt generation, communication with a generative AI model, and control of an information processing apparatus.

The term “sensor apparatus” refers to a hardware unit including one or more sensing elements configured to measure biological signals of a subject, such as physiological state information including at least heart rate information and blood pressure information, and to output corresponding measurement data.

The term “biological information” refers to data representing a physical condition of a subject, including physiological state information such as heart rate information, blood pressure information, body temperature information, or other biometric measurements acquired by the sensor apparatus.

The term “physiological state information” refers to a subset of biological information that quantitatively represents a physiological state of a subject, such as heart rate information, blood pressure information, respiratory rate information, or skin conductance information.

The term “heart rate information” refers to data indicating the number of heart beats of a subject per unit time, including instantaneous values, average values, or derived statistics of heart activity.

The term “blood pressure information” refers to data indicating arterial pressure of a subject, including at least systolic pressure and diastolic pressure values or derived metrics based on these values.

The term “behavior information” refers to data representing actions or motions of a subject, including but not limited to body movement, posture, gaze direction, or interaction patterns, which may be obtained from motion sensors, image sensors, or other detectors.

The term “emotion information” refers to data indicating or suggesting an emotional state of a subject, such as happiness, sadness, anxiety, or stress, derived from one or more modalities including text, voice characteristics, facial expressions, or physiological responses.

The term “subject” refers to a human individual whose biological information, behavior information, and/or emotion information are acquired and processed by the system.

The term “analysis condition” refers to contextual parameters, constraints, or instructions included in a prompt sentence that specify how a generative AI model is to analyze input data, such as requested output format, classification categories, or evaluation criteria.

The term “state of the subject” refers to a representation of a physical and mental state or an emotional state of the subject, such as relaxed, stressed, fatigued, or anxious, which is derived from acquired information and may be included in a prompt sentence.

The term “usage context” refers to information describing an operational situation in which the system is used, such as a retail environment, workplace environment, or home environment, which is included in a prompt sentence to provide situational background for the generative AI model.

The term “prompt sentence” refers to a sequence of characters expressed in natural language that encodes an instruction, query, or description, and is supplied as input to a generative AI model to cause the generative AI model to produce an output.

The term “first prompt sentence” refers to a prompt sentence generated based on biological information and at least one of behavior information and emotion information, and configured to request from a generative AI model an analysis result or an action candidate relating to a state of the subject.

The term “control prompt sentence” refers to a prompt sentence expressed in natural language that specifies an operation content of an information processing apparatus, the control prompt sentence being generated based on an analysis result or an action candidate and used to control an operation of the information processing apparatus.

The term “second prompt sentence” refers to a prompt sentence that instructs a generative AI model to generate an action content related to support, encouragement, or consideration for a human, and that is used to obtain a natural language message corresponding to a state of the subject.

The term “generative AI model” refers to a machine learning model configured to generate natural language text or other data in response to a prompt sentence, by using learned parameters obtained from training on large amounts of data, and which is capable of performing reasoning or analysis based on the prompt sentence.

The term “analysis result” refers to an output produced by a generative AI model or another analysis process, indicating at least one of an evaluation, classification, estimation, or explanation relating to a physical and mental state or an emotional state of a subject.

The term “action candidate” refers to an output produced by a generative AI model that describes one or more proposed actions, operations, or responses to be taken by an information processing apparatus, an operator, or another component in view of a state of a subject.

The term “information processing apparatus” refers to a hardware and/or software system that performs data processing, control, or presentation functions, and includes at least one of an environment control apparatus and an information presentation apparatus.

The term “environment control apparatus” refers to a device or subsystem that adjusts a physical environment around a subject, such as lighting, sound, temperature, or visual displays, in response to control instructions.

The term “information presentation apparatus” refers to a device or subsystem that presents information to a subject or to an operator, including at least one of a display apparatus and an audio output apparatus.

The term “display apparatus” refers to a hardware unit that visually presents information, such as a monitor, head-mounted display, smart glasses display, or mobile device screen.

The term “audio output apparatus” refers to a hardware unit that outputs sound, such as a loudspeaker, earphone, or headset, to present audio information including synthesized speech.

The term “support message” refers to a natural language message generated based on an analysis result or an action candidate, the message being intended to provide guidance, support, encouragement, or consideration to a subject.

The term “proposal content” refers to information indicating recommended actions, services, or items to be suggested to a subject or an operator, generated using a natural language response obtained from a generative AI model.

The term “attribute information” refers to static or slowly changing information about a subject, such as demographic information, preference information, or role information, which can be used as context when generating messages or control instructions.

The term “history information” refers to time-series or accumulated information associated with past states, interactions, or system outputs for a subject, including previous biological information, previous analysis results, and previous system actions.

The term “presentation apparatus” refers to an apparatus that provides information to a subject or an operator and includes at least one of a display apparatus and an audio output apparatus.

The term “operator” refers to a human individual, different from the subject in some use cases, who receives information from the system and performs actions such as assisting or managing the subject.

The term “response information” refers to information indicating a reaction or feedback from a subject, such as verbal responses, textual input, behavior changes, or explicit selections, which is used to update prompt sentences and control logic.

The term “feature quantity” refers to a numerical or categorical value derived from raw physiological state information or other input data, computed by processing such as aggregation, normalization, or statistical analysis, and used as a compact representation for analysis or prompt construction.

The term “iteratively optimize” refers to a process in which the processor repeatedly updates parameters, prompt sentence components, or control policies based on newly acquired data and past outputs, in order to improve performance metrics such as responsiveness, accuracy, or appropriateness of actions over time.

In one embodiment, a server implements the claimed system by executing a computer program on a hardware platform that includes at least one processor, a main memory, a non-volatile storage device, and a network interface. The server runs an operating system such as a general-purpose server operating system and executes application software modules written, for example, in a scripting language and using libraries for numerical computation, machine learning, and network communication. The server communicates with one or more terminals over a network such as a wireless local network or a public data network. Each terminal includes at least one sensor apparatus, such as a wearable device with heart-rate and blood-pressure sensors, a camera, a microphone, or a motion sensor, and further includes at least one presentation apparatus such as a display and an audio output device. A user wears or operates the terminal so that biological information, behavior information, and emotion-related information can be acquired.

The server configures a data acquisition module to receive biological information from the terminals. The terminal acquires raw sensor readings, such as heart-rate values, blood-pressure values, body temperature values, or skin-conductance values, at a predetermined sampling interval. The terminal encapsulates each reading with associated metadata, including a subject identifier, a device identifier, and a timestamp, and transmits these data items to the server using a secure application-layer protocol. The server stores incoming records in a structured data repository, such as a relational database, using a schema designed to separate raw measurements from derived features. The server thereby maintains ordered sequences of biological information for each subject, which enables time-series processing and correlation with subsequent analysis and control operations.

The server configures a preprocessing module to transform raw biological information into feature quantities suitable for downstream analysis and for inclusion in prompt sentences. The server, using numerical computation libraries, executes operations such as outlier detection based on predetermined physiological thresholds, interpolation of missing samples by linear interpolation or spline interpolation, and smoothing of high-frequency noise by applying moving-average filters or low-pass digital filters. The server aggregates raw data over fixed time windows to compute statistical feature quantities, such as mean heart rate, heart-rate variability indices based on standard deviation of inter-beat intervals, average systolic and diastolic blood pressure, and variance of skin-conductance values. The server normalizes these feature quantities, for example, by z-score normalization relative to a baseline for the subject, and stores the results in a feature table that is indexed by subject and time interval. This preprocessing and feature extraction reduce data dimensionality and noise, thereby improving computational efficiency and stability of subsequent machine-learning operations.

The server further configures an emotion-analysis module to derive behavior information and emotion information. The terminal acquires image data from a camera and audio data from a microphone when the user interacts with the system. The terminal compresses image frames and audio segments and transmits them to the server. The server applies, for example, a computer-vision library to detect facial landmarks and compute facial expression features such as eyebrow position, mouth curvature, and eye openness. The server also applies a speech-analysis library to compute prosodic features from audio, such as fundamental frequency, energy, and speaking rate. The server combines these features with textual content obtained from a speech-to-text engine or direct text input from the user. The server then executes a multi-modal classifier, which may be implemented as a neural network with separate sub-networks for visual, acoustic, and textual features and a fusion layer, to output emotion labels such as “relaxed,” “stressed,” or “anxious” with associated probabilities. This classifier can use, for instance, a convolutional sub-network for image features, a recurrent or transformer-based sub-network for sequential audio and text features, and a fully connected layer for final classification. The server stores the resulting emotion information in association with the feature quantities of the biological information.

The server implements a local state-estimation model in addition to the generative AI model. The server loads a trained neural network that receives as input a vector consisting of time-windowed physiological feature quantities and optionally encoded emotion features. The network may be a feed-forward multilayer perceptron with several hidden layers, or a recurrent neural network capable of modeling temporal dependencies. During training, the server can use back-propagation with an objective function such as cross-entropy loss for categorical stress levels or mean-squared error for continuous stress scores, and can update weight parameters using an optimization method such as stochastic gradient descent with momentum or an adaptive gradient method. To increase robustness, the server may apply data augmentation techniques at training time, such as adding small variations to physiological values within clinically plausible ranges. At runtime, the server executes only the forward-propagation part of the network to compute internal state estimates, which reduces latency and computational cost compared with maintaining multiple separate rule-based modules.

The server integrates these internal analysis components with a generative AI model through the use of prompt sentences. The server configures a prompt-generation module that constructs a first prompt sentence in natural language from structured data. The server retrieves, for a given subject and current time interval, feature quantities (for example, mean heart rate, heart-rate variability, and blood pressure), internal state estimates (for example, a continuous stress score), emotion labels, and usage context (such as “retail store” or “office workplace”). The server converts numerical values to human-readable form using controlled rounding and units, such as beats per minute or millimeters of mercury, and includes them in template text. For example, the server may generate a first prompt sentence such as:

“The user is in a retail store. Current heart rate is 110 bpm, up from a baseline of 75 bpm. Blood pressure is 145 over 95. Recent facial expression and voice tone suggest tension. Classify the stress level as low, medium, or high, explain briefly, and propose two concrete actions that staff can take.”

The server supplies this first prompt sentence to a generative AI model hosted either on the same server or on a remote computation service. The generative AI model can be implemented as a large transformer neural network trained on large-scale corpora and fine-tuned for instruction following. Internally, this model uses multi-head self-attention layers, layer-normalization, and positional encodings to process token sequences representing the prompt sentence and to generate output tokens that are decoded into natural-language text. The server invokes the model through a programmatic interface, specifying parameters such as maximum output length, temperature for sampling, and whether to require a particular response format, which allows the server to balance diversity and determinism of the output.

The server receives from the generative AI model an analysis result and candidate actions encoded in natural language. For example, the model may produce an output such as:
“Stress level is high. Staff should guide the customer to a quieter area and offer products that promote relaxation, such as herbal drinks or aroma devices.”

The server parses the received text using rule-based patterns or lightweight natural-language parsing. The server identifies key segments such as the stress classification and the recommended actions by detecting keywords and phrase structures, and maps them into structured data fields. This structured representation enables the server to integrate the generative AI output with control logic for environment-control apparatuses and information-presentation apparatuses in a deterministic manner.

In another embodiment, the server generates a control prompt sentence specifically for controlling an information processing apparatus. The server uses the analysis result or action candidate to construct a control prompt sentence that is still expressed in natural language but follows a restricted format understood by a controller module. For instance, the server may generate:
“Set the ambient light level to 50 percent, switch the color tone to warm, and start a calm instrumental music playlist at low volume.”

The server supplies this control prompt sentence to a control-translation component that maps specific phrases to device-level commands. The control-translation component can maintain a dictionary of phrases and corresponding command codes for environment-control apparatuses such as lighting controllers, audio playback systems, or display systems. The server then sends the resulting command codes via device-specific protocols, such as a local network protocol for a lighting gateway or a web-based application programming interface for an audio system. In this way, the natural-language control prompt sentence serves as an intermediate representation that simplifies integration of new devices and allows dynamic behavior without rewriting low-level control code.

The server also generates a second prompt sentence to obtain support messages or proposal content from the generative AI model. The server includes in the second prompt sentence information such as the subject's current state, a summary of recent history, and constraints on message length and tone. For example, the server may generate:

“The user has been working for 6 hours with a stress level of 0.8 on a scale from 0 to 1. Generate a short, caring message, within 30 words, encouraging the user to take a 5-minute break and do simple stretching exercises.”

The generative AI model outputs a natural-language message that the server passes to the terminal for presentation to the subject through a display or converted into speech using a text-to-speech engine. Because the server centrally constructs and updates the prompt sentences based on continuously acquired biological information and response information from the user, the content of these messages adapts in real time to changes in the user's state without requiring static rule sets for each possible condition.

The server updates prompt-sentence components iteratively. The server monitors sequences of biological information and response information, such as whether the user follows suggestions, how quickly physiological parameters change after interventions, and how the user's emotion information evolves. The server computes feedback metrics, such as reduction in stress score or frequency of positive emotional labels, and adjusts the structure of future prompt sentences, for example by adding more precise constraints or by narrowing the requested action space. This iterative prompt optimization reduces the computational load on the generative AI model by focusing generation on relevant alternatives, and reduces network traffic by limiting prompt and response lengths while maintaining sufficient context for accurate reasoning.

The described architecture improves computer technology in several ways. By representing both analysis requests and control instructions as structured prompt sentences that encode multi-modal state and context, the server reduces the need for multiple, separate decision modules and complex interface code between them. The server uses feature extraction and normalized summaries of time-windowed biological information, which enables smaller prompts with high information content and reduces communication overhead between server and generative AI model. The use of a unified generative AI model for both analysis and action recommendation allows the server to remove duplicated logic, leading to lower memory usage and reduced latency compared with systems that run separate rule engines. Furthermore, the prompt-generation and parsing pipeline is designed as a non-conventional orchestration layer that uses specific templates, keyword markers, and structured segments, which enables deterministic mapping from natural-language outputs to device commands, thereby overcoming the unpredictability associated with generic language-generation systems.

The system is not limited to a single use scenario. In one variation, the server cooperates with terminals deployed in a factory environment. The sensor apparatus includes cameras observing workers, and the environment-control apparatus includes speakers and indicator lights near workstations. The server constructs prompt sentences that describe detected posture, motion patterns, and derived fatigue levels, and asks the generative AI model to propose concise and safe voice messages. In another variation, the server operates in a home-health context, where the sensor apparatus is a wearable device measuring sleep patterns in addition to heart rate and blood pressure, and the information-presentation apparatus is a mobile device. The same prompt-based orchestration is applied, but templates are adapted to nighttime monitoring and longer-term trends. In further variations, the generative AI model may be replaced by another generative model with similar capabilities, or may run locally on the server hardware for scenarios with restricted network connectivity.

The terminal functions as an interface between the subject and the server. The terminal acquires data from local sensor apparatuses, formats the data according to a predefined message structure that includes fields for subject identity, device type, and measurement values, and transmits the data to the server. The terminal also receives support messages, proposal content, and summary information such as stress levels from the server, and presents them to the user via a display, a head-mounted display, or an audio output device. In some embodiments, the terminal executes local preprocessing to reduce bandwidth, such as aggregating high-frequency sensor readings before transmission, or compressing image frames using lossy compression with a controlled quality factor.

The user interacts with the system by wearing or carrying the terminal, by providing explicit input through text or speech, and by responding to feedback such as messages or environmental changes. The user's behavior and responses are captured as additional input to the server, which allows the system to refine its internal models and prompt structures. The arrangement of server, terminal, and user enables continuous sensing and adaptive feedback loops that cannot be achieved with purely static or rule-based approaches.

By combining structured feature extraction, prompt-based orchestration of a generative AI model, and deterministic translation of generative outputs into device commands, the system provides a technical solution that improves the functioning of the computer system itself. The server performs fewer redundant computations, manages multi-modal data more efficiently, and controls external apparatuses in a more responsive and accurate manner than conventional architectures in which analysis, decision, and control logic are fragmented. The described embodiments therefore enable practical implementation of the claimed invention across various environments while providing clear technical effects such as improved processing speed, reduced communication load, enhanced state-estimation accuracy, and more reliable control of environment-control and information-presentation apparatuses.

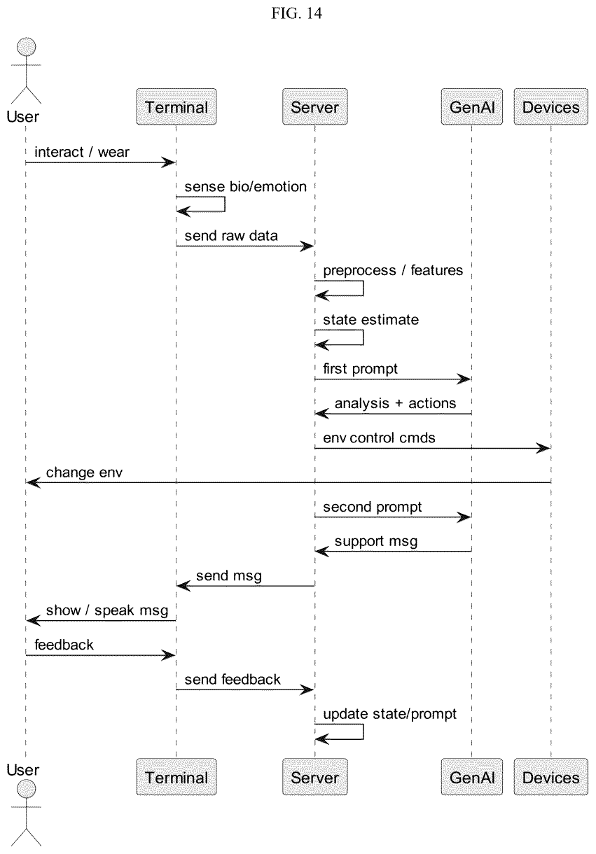

The following describes the processing flow using FIG. 14.

### Step 1:

Terminal acquires raw sensor data from the user.

Terminal uses one or more sensor apparatuses (for example, heart-rate sensor, blood-pressure sensor, accelerometer, camera, microphone) to measure biological information and behavior information of the user at fixed sampling intervals.

Input: physical state of the user measured by the sensors.

Output: a raw data record including sensor type, measured value (for example, heartRate=110, bloodPressure=145/95), timestamp, and device identifier.

Terminal packages these values into a structured message and buffers them in local memory for transmission.

### Step 2:

Terminal transmits raw data records to the server.

Terminal opens a secure network connection and sends each buffered record to the server using a predefined application protocol.

Input: locally stored raw data records generated in Step 1.

Output: network messages containing encoded raw data records delivered to the server. Terminal may batch multiple records into a single transmission to reduce communication overhead, and appends a sequence number to each batch for integrity checking.

### Step 3:

Server receives, validates, and stores raw data records.

Server listens on a network interface for incoming messages from terminals and decodes the message format to extract individual records.

Input: network messages containing raw sensor data from one or more terminals.

Output: validated raw records stored in a database table with fields for subject ID, device ID, sensor type, value, and timestamp.

Server checks that required fields are present, that values fall within allowable numeric ranges, and that timestamps are in chronological order; server discards or flags records that fail validation.

### Step 4:

Server preprocesses raw biological information and computes feature quantities.

Server reads newly stored raw records over a predefined time window (for example, the last 60 seconds) for each subject and applies numerical processing.

Input: sets of raw sensor values and associated timestamps for a subject.

Output: a feature vector including aggregated and normalized quantities, such as mean heart rate, heart-rate variability, average systolic and diastolic pressure, and motion level.

Server performs data processing such as outlier removal using threshold rules, interpolation of short gaps by linear interpolation, smoothing by moving-average filtering, and aggregation by computing means, variances, and slopes over the time window.

### Step 5:

Terminal acquires user interaction data for emotion analysis.

Terminal captures images from a camera and audio from a microphone while the user is present, and optionally collects text typed by the user.

Input: current visual appearance and spoken or typed expressions of the user.

Output: encoded image frames, audio segments, and text strings with timestamps and subject identifiers.

Terminal compresses images and audio to a target quality level, associates them with metadata, and transmits them to the server.

### Step 6:

Server derives behavior information and emotion information.

Server processes the received images and audio to compute behavior- and emotion-related features and then classifies emotion states.

Input: encoded image frames, audio segments, and text strings from the terminal.

Output: emotion labels (for example, “relaxed,” “stressed,” “anxious”) with confidence values and behavior descriptors (for example, “slouched posture,” “rapid speech”).

Server applies a vision module to detect facial landmarks and expression parameters, a speech module to extract prosodic features such as pitch and energy, and a text-analysis module to compute sentiment scores; server then feeds these features into a multi-modal classifier to obtain emotion and behavior labels.

### Step 7:

Server estimates an internal state of the user from biological and emotion features.

Server combines feature quantities from Step 4 with emotion information from Step 6 and runs a trained neural-network model to estimate a physical and mental state score.

Input: numerical feature vector of physiological data and encoded emotion features.

Output: state estimates such as stressLevel, fatigueLevel, or relaxationLevel expressed as continuous values or category probabilities.

Server performs forward-propagation through a multi-layer neural network, executing matrix multiplications and non-linear activation functions to compute state scores from the input vector.

### Step 8:

Server constructs a first prompt sentence for the generative AI model.

Server converts the structured state information, feature values, and context information into a natural-language instruction sequence.

Input: state estimates, feature quantities (for example, mean heart rate, blood pressure), emotion labels, and usage context (for example, “retail store,” “office”).

Output: a first prompt sentence expressed in natural language that describes the current situation and requests analysis and action candidates.

Server selects a template, inserts numerical values with units, encodes context phrases, and appends explicit instructions such as “classify the stress level” and “propose two concrete actions.”

### Step 9:

Server sends the first prompt sentence to the generative AI model and receives an analysis result.

Server invokes an external or internal generative AI service, passing the prompt and controlling output parameters.

Input: the first prompt sentence generated in Step 8.

Output: a natural-language response containing an analysis result and proposed action candidates.

Server transmits the prompt as a sequence of tokens to the generative AI model, which executes attention-based transformations across multiple layers to generate response tokens; server decodes the tokens back into text and stores the resulting response.

### Step 10:

Server parses the generative AI response into structured analysis and action candidates.

Server processes the returned text to extract specific fields and decision elements required for control.

Input: the natural-language response text from the generative AI model.

Output: structured data items including a stress classification, explanatory text, and one or more action candidates with textual descriptions.

Server applies rule-based matching, key-phrase extraction, and simple pattern recognition to segment the response into labeled sections, and converts relevant phrases into structured records.

### Step 11:

Server generates a control prompt sentence specifying operations of information processing apparatuses.

Server composes a natural-language control instruction that maps the analysis result and action candidates to concrete device-level goals.

Input: structured analysis data and action candidates from Step 10.

Output: a control prompt sentence describing desired environment changes or information-presentation actions.

Server applies a control template such as “Set light level to X, change color to Y, play Z type of music,” and fills in device-independent values (for example, 50 percent brightness, warm color tone, calm instrumental music) derived from the action candidates.

### Step 12:

Server translates the control prompt sentence into device commands and transmits them.

Server uses a control-translation table to convert segments of the control prompt into device-specific command codes and then sends these codes to the target apparatuses.

Input: the control prompt sentence generated in Step 11.

Output: device-specific control messages sent to environment-control apparatuses and information-presentation apparatuses.

Server tokenizes the control prompt, matches key phrases against a mapping database, populates command structures (for example, JSON objects or binary command frames), and transmits them via corresponding communication protocols to lighting controllers, audio systems, or display controllers.

### Step 13:

Terminal receives control messages and performs environment or presentation actions.

Terminal, acting as a local controller or gateway, accepts control messages from the server and applies them to connected devices.

Input: device-specific control messages from the server.

Output: actual changes in physical environment or displayed information, such as adjusted lighting, started music playback, or updated on-screen content.

Terminal parses the received messages, sets hardware registers or calls device APIs for each controlled apparatus, and monitors status feedback to confirm successful execution.

### Step 14:

Server generates a second prompt sentence to obtain a support message or proposal content.

Server prepares another natural-language instruction focused on generating a message for the user or an operator.

Input: current state estimates, recent history values, and, optionally, results of the environment changes already applied.

Output: a second prompt sentence instructing the generative AI model to create a short, context-appropriate message.

Server assembles the prompt by describing the user's condition and specifying constraints such as style, length, and purpose (for example, “encourage the user to take a short break”).

### Step 15:

Server requests a support message from the generative AI model and receives the result.

Server sends the second prompt sentence to the generative AI model and waits for a natural-language message in response.

Input: the second prompt sentence generated in Step 14.

Output: a natural-language support message or proposal content tailored to the user's state.

Server forwards the prompt to the generative AI engine, receives the generated text, and stores it together with the associated state estimates for logging and later analysis.

### Step 16:

Terminal presents the support message or proposal content to the user or operator.

Terminal obtains the message text from the server and chooses an appropriate presentation modality based on terminal capabilities.

Input: the natural-language support message or proposal content from the server.

Output: a visual or auditory representation of the message visible or audible to the user or operator.

Terminal displays the text on a screen, overlays it on a head-mounted display, or converts it into audio using a text-to-speech engine and plays it through speakers or headphones.

### Step 17:

User responds to feedback and optionally provides additional input.

User reacts to the presented message and environment changes, for example by following a suggested action or by explicitly answering with text or speech.

Input: feedback information received by the user from the terminal (messages, environment changes).

Output: behavioral changes (for example, taking a break, relaxing posture) and/or explicit responses (spoken statements, typed text).

User's behavior is captured indirectly by the sensor apparatus, and explicit responses are captured by microphone or text interface on the terminal.

### Step 18:

Server updates state and prompt parameters based on continuous sensing and user response.

Server retrieves new biological information and response information and recalculates state estimates and performance metrics for past actions.

Input: newly acquired sensor data, updated emotion information, and indicators of whether suggested actions were followed or effective.

Output: adjusted model inputs, updated user state records, and modified parameters or templates for future prompt sentences.

Server compares new feature quantities and emotion labels with previous values, computes differences (for example, reduction in stress score), and modifies prompt templates—for example, by changing requested action types or tightening classification criteria—to iteratively optimize subsequent interactions with the generative AI model and the control of the information processing apparatus.

The data generation model 58 is a so-called generative artificial intelligence (AI). Examples of the data generation model 58 include generative AIs such as ChatGPT (registered trademark) (Internet search <URL: https://openai.com/blog/chatgpt>) and the like. The data generation model 58 is obtained by performing deep learning with a neural network. The data generation model 58 is input with a prompt including an instruction, and is input with inference data such as audio data representing speech, text data representing text, image data representing images (for example, still image data or video data), and the like. The data generation model 58 takes the input inference data, performs inference according to the instruction indicated in the prompt, and outputs an inference result in one or more data format from out of audio data, text data, image data, or the like. The data generation model 58 includes, for example, a text generative AI, an image generative AI, a multimodal generative AI, or the like. Reference here to inference indicates, for example, analysis, classification, prediction, and/or abstraction etc. The specific processing unit 290 performs the specific processing referred to above while using the data generation model 58. The data generation model 58 may be a model fine-tuned so as to output an inference result from a prompt not including an instruction, and in such cases the data generation model 58 is able to output an inference result from the prompt not including an instruction. There are plural types of the data generation model 58 included in the data processing device 12 or the like, and the data generation models 58 include an AI other than a generative AI. An AI other than a generative AI is, for example, a linear regression, a logistic regression, a decision tree, a random forest, a support vector machine (SVM), a k-means clustering, a convolutional neural network (CNN), a recurrent neural network (RNN), a generative adversarial network (GAN), a naïve Bayes, or the like and is capable of performing various processing, however there is no limitation to such examples. The AI may be an AI agent. Moreover, when the processing of each of the units mentioned above is performed by an AI, this processing is partly or entirely performed by the AI, however there is no limitation to such examples. Moreover, processing executed by an AI including a generative AI may be switched to rule-based processing, and rule-based processing may be switched to processing executed by an AI including a generative AI.

Moreover, although the processing by the data processing system 10 described above was executed by the specific processing unit 290 of the data processing device 12 or by the control unit 46A of the smart device 14, the processing may be executed by a specific processing unit 290 of the data processing device 12 and a control unit 46A of the smart device 14. Moreover, the specific processing unit 290 of the data processing device 12 acquires and collects information needed for processing from the smart device 14 or from an external device or the like, and the smart device 14 acquires and collects information needed for processing from the data processing device 12 or from an external device or the like.

For example, a collection unit is implemented by the control unit 46A of the smart device 14 and/or by the specific processing unit 290 of the data processing device 12. For example, an acquisition unit acquires number-of-steps data using the camera 42 and/or the communication I/F 44 of the smart device 14, and the number-of-steps data is processed by the specific processing unit 290 of the data processing device 12. For example, an analysis unit implemented by the specific processing unit 290 of the data processing device 12 analyzes data from the collection unit and the acquisition unit. For example, a generation unit implemented by the specific processing unit 290 of the data processing device 12 generates a cooking menu using a generative AI. For example, a supply unit implemented by the output device 40 of the smart device 14 and/or the specific processing unit 290 of the data processing device 12 supplies the generated cooking menu to the user. Correspondence relationships of each unit to devices and control units are not limited to the examples described above, and various modifications thereof are possible.

The above exemplary embodiment gives an implementation example in which the specific processing is performed by the data processing device 12, however technology disclosed herein is not limited thereto, and the specific processing may be performed by the smart device 14.

### Second Exemplary Embodiment

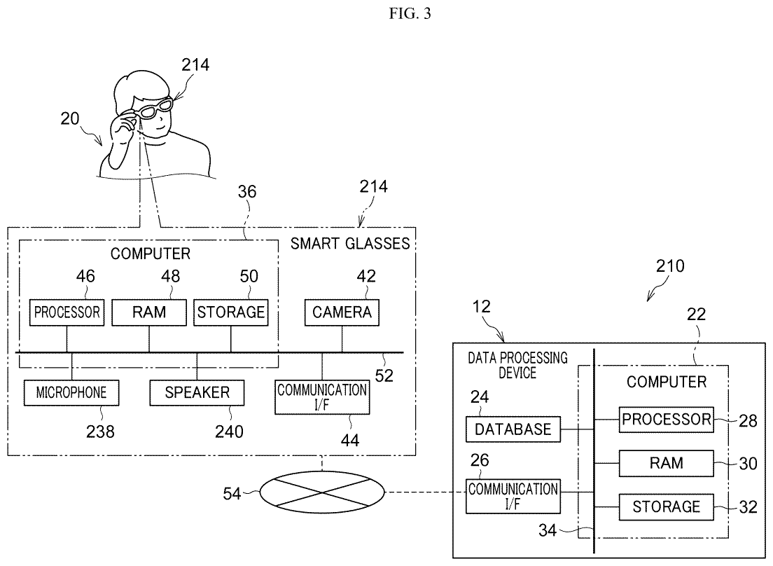

FIG. 3 illustrates an example of a configuration of a data processing system 210 according to a second exemplary embodiment.

As illustrated in FIG. 3, the data processing system 210 includes a data processing device 12 and smart glasses 214. A server is an example of the data processing device 12.

The data processing device 12 includes a computer 22, a database 24, and a communication I/F 26. The computer 22 is an example of a “computer” according to technology disclosed herein. The computer 22 includes a processor 28, RAM 30, and storage 32. The processor 28, the RAM 30, and the storage 32 are connected to a bus 34. The database 24 and the communication I/F 26 are also connected to the bus 34. The communication I/F 26 is connected to a network 54. Examples of the network 54 include a Wide Area Network (WAN) and/or a local area network (LAN).

The smart glasses 214 include a computer 36, a microphone 238, a speaker 240, a camera 42, and a communication I/F 44. The computer 36 includes a processor 46, RAM 48, and storage 50. The processor 46, the RAM 48, and the storage 50 are connected to a bus 52. The microphone 238, the speaker 240, the camera 42, and the communication I/F 44 are also connected to the bus 52.

The microphone 238 receives an instruction or the like from a user 20 by receiving speech uttered by the user 20. The microphone 238 captures the speech uttered by the user 20, converts the captured speech into audio data, and outputs the audio data to the processor 46. The speaker 240 outputs audio under instruction from the processor 46.

The camera 42 is a compact digital camera installed with an optical system such as a lens, an aperture, a shutter, and the like, and with an imaging device such as a complementary metal-oxide semiconductor (CMOS) image sensor or a charge coupled device (CCD) image sensor or the like. The camera 42 images the surroundings of the user 20 (for example, an imaging range defined by an angle of view equivalent to the width of visual field of an ordinary healthy subject).

The communication I/F 44 is connected to the network 54. The communication I/F 44 and the communication I/F 26 perform the role of exchanging various information between the processor 46 and the processor 28 over the network 54. The exchange of various information between the processor 46 and the processor 28 is performed in a secure state using the communication I/F 44 and the communication I/F 26.

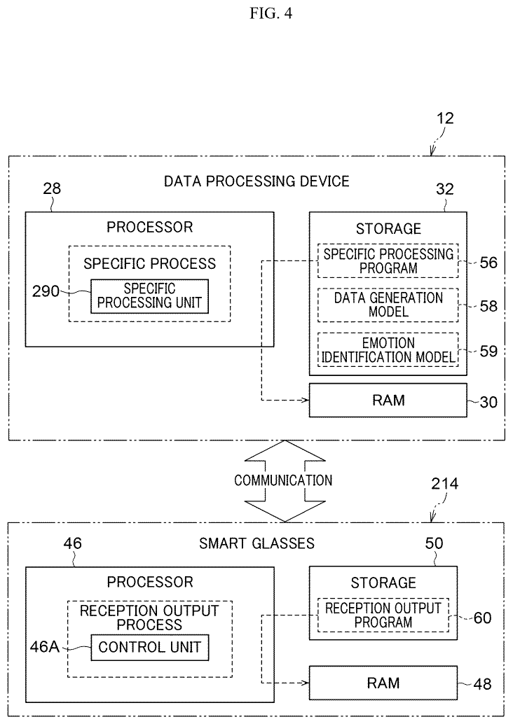

FIG. 4 illustrates an example of relevant functions of the data processing device 12 and the smart glasses 214. As illustrated in FIG. 4, specific processing is performed by the processor 28 in the data processing device 12. A specific processing program 56 is stored in the storage 32.

The specific processing program 56 is an example of a “program” according to technology disclosed herein. The processor 28 reads the specific processing program 56 from the storage 32, and in the RAM 30 executes the read specific processing program 56. The specific processing is implemented by the processor 28 operating as the specific processing unit 290 according to the specific processing program 56 executed in the RAM 30.

The data generation model 58 and the emotion identification model 59 are stored in the storage 32. The data generation model 58 and the emotion identification model 59 are employed by the specific processing unit 290. The specific processing unit 290 uses the emotion identification model 59 to estimate an emotion of a user, and is able to perform the specific processing using the user emotion. In an emotion estimation function (emotion identification function) that uses the emotion identification model 59, various estimations, predictions, and the like are performed related to emotions of the user, include estimating and predicting the emotion of the user, however, there is no limitation to such examples. Moreover, estimation and prediction of emotion also includes, for example, analyzing (parsing) emotions and the like.

Reception and output processing is performed by the processor 46 in the smart glasses 214. A reception and output program 60 is stored in the storage 50. The processor 46 reads the reception and output program 60 from the storage 50 and in the RAM 48 executes the read reception and output program 60. The reception and output processing is implemented by the processor 46 operating as the control unit 46A according to the reception and output program 60 executed in the RAM 48. Note that a configuration may be adopted in which the smart glasses 214 include a data generation model and an emotion identification model similar to the data generation model 58 and the emotion identification model 59, and processing similar to the specific processing unit 290 is performed using these models.

Next, description follows regarding the specific processing by the specific processing unit 290 of the data processing device 12. The units of the system described below are implemented by the data processing device 12 and the smart glasses 214. In the following description the data processing device 12 is called a “server”, and the smart glasses 214 is called a “terminal”.

### Example 1

Explanation of flow will be omitted due to being similar to a flow of the specific processing in Example 1 as described in the first exemplary embodiment above.

### Application Example 1

Explanation of flow will be omitted due to being similar to a flow of the specific processing in Application Example 1 as described in the first exemplary embodiment above.

### Example 2

Explanation of flow will be omitted due to being similar to a flow of the specific processing in Example 2 as described in the first exemplary embodiment above.

### Application Example 2

Explanation of flow will be omitted due to being similar to a flow of the specific processing in Application Example 2 as described in the first exemplary embodiment above.

The specific processing unit 290 transmits a result of the specific processing to the smart glasses 214. The control unit 46A in the smart glasses 214 outputs the specific processing result to the speaker 240. The microphone 238 acquires audio representing user input in response to the specific processing result. The control unit 46A transmits audio data representing the user input as acquired by the microphone 238 to the data processing device 12. The specific processing unit 290 in the data processing device 12 acquires the audio data.

The data generation model 58 is a so-called generative artificial intelligence (AI). Examples of the data generation model 58 include generative Als such as ChatGPT (registered trademark) (Internet search <URL: https://openai.com/blog/chatgpt>) and the like. The data generation model 58 is obtained by performing deep learning with a neural network. The data generation model 58 is input with a prompt including an instruction, and is input with inference data such as audio data representing speech, text data representing text, image data representing images (for example, still image data or video data), and the like. The data generation model 58 takes the input inference data, performs inference according to the instruction indicated in the prompt, and outputs an inference result in one or more data format from out of audio data, text data, image data, or the like. The data generation model 58 includes, for example, a text generative AI, an image generative AI, a multimodal generative AI, or the like. Reference here to inference indicates, for example, analysis, classification, prediction, and/or abstraction etc. The specific processing unit 290 performs the specific processing referred to above while using the data generation model 58. The data generation model 58 may be a model fine-tuned so as to output an inference result from a prompt not including an instruction, and in such cases the data generation model 58 is able to output an inference result from the prompt not including an instruction. There are plural types of the data generation model 58 included in the data processing device 12 or the like, and the data generation models 58 include an AI other than a generative AI. An AI other than a generative AI is, for example, a linear regression, a logistic regression, a decision tree, a random forest, a support vector machine (SVM), a k-means clustering, a convolutional neural network (CNN), a recurrent neural network (RNN), a generative adversarial network (GAN), a naïve Bayes, or the like and is capable of performing various processing, however there is no limitation to such examples. The AI may be an AI agent. Moreover, when the processing of each of the units mentioned above is performed by an AI, this processing is partly or entirely performed by the AI, however there is no limitation to such examples. Moreover, processing executed by an AI including a generative AI may be switched to rule-based processing, and rule-based processing may be switched to processing executed by an AI including a generative AI.

Although the processing by the data processing system 10 described above is executed by the specific processing unit 290 of the data processing device 12 or by the control unit 46A of the smart glasses 214, the processing may be executed by a specific processing unit 290 of the data processing device 12 and a control unit 46A of the smart glasses 214. Moreover, the specific processing unit 290 of the data processing device 12 acquires and collects information needed for processing from the smart glasses 214 or from an external device or the like, and the smart glasses 214 acquires and collects information needed for processing from the data processing device 12 or from an external device or the like.

For example, the collection unit is implemented by the control unit 46A of the smart glasses 214 and/or by the specific processing unit 290 of the data processing device 12. For example, an acquisition unit acquires number-of-steps data using the camera 42 and/or the communication I/F 44 of the smart glasses 214, and the number-of-steps data is processed by the specific processing unit 290 of the data processing device 12. For example, an analysis unit implemented by the specific processing unit 290 of the data processing device 12 analyzes data from the collection unit and the acquisition unit. For example, a generation unit implemented by the specific processing unit 290 of the data processing device 12 generates a cooking menu using a generative AI. For example, a supply unit implemented by the speaker 240 of the smart glasses 214 and/or the specific processing unit 290 of the data processing device 12 supplies the generated cooking menu to the user. Correspondence relationships of each unit to devices and control units are not limited to the examples described above, and various modifications thereof are possible.

The above exemplary embodiment gives an implementation example in which the specific processing is performed by the data processing device 12, however technology disclosed herein is not limited thereto, and the specific processing may be performed by the smart glasses 214.

### Third Exemplary Embodiment

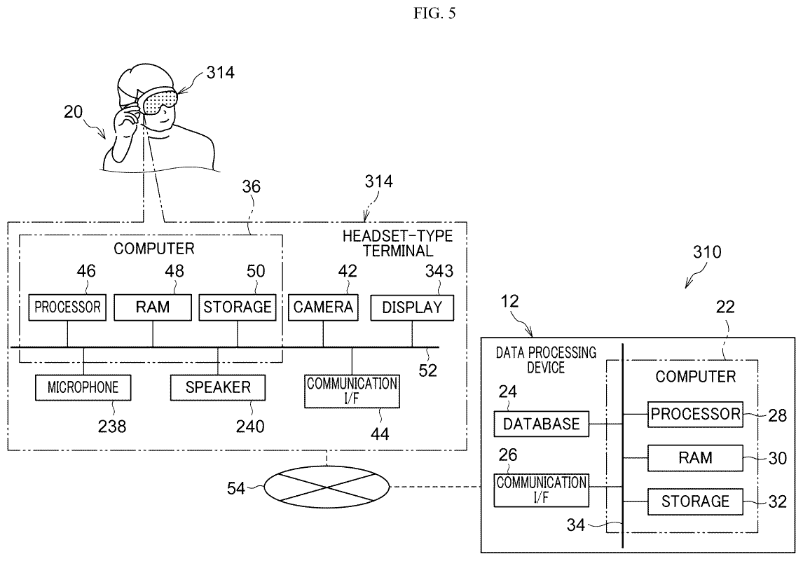

FIG. 5 illustrates an example of a configuration of a data processing system 310 according to a third exemplary embodiment.

As illustrated in FIG. 5, the data processing system 310 includes a data processing device 12 and a headset-type terminal 314. A server is an example of the data processing device 12.

The data processing device 12 includes a computer 22, a database 24, and a communication I/F 26. The computer 22 is an example of a “computer” according to technology disclosed herein. The computer 22 includes a processor 28, RAM 30, and storage 32. The processor 28, the RAM 30, and the storage 32 are connected to a bus 34. The database 24 and the communication I/F 26 are also connected to the bus 34. The communication I/F 26 is connected to a network 54. Examples of the network 54 include a Wide Area Network (WAN) and/or a local area network (LAN).

The headset-type terminal 314 includes a computer 36, a microphone 238, a speaker 240, a camera 42, a communication I/F 44, and a display 343. The computer 36 includes a processor 46, RAM 48, and storage 50. The processor 46, the RAM 48, and the storage 50 are connected to a bus 52. The microphone 238, the speaker 240, the camera 42, the display 343, and the communication I/F 44 are also connected to the bus 52.

The microphone 238 receives an instruction or the like from a user 20 by receiving speech uttered by the user 20. The microphone 238 captures the speech uttered by the user 20, converts the captured speech into audio data, and outputs the audio data to the processor 46. The speaker 240 outputs audio under instruction from the processor 46.

The camera 42 is a compact digital camera installed with an optical system such as a lens, an aperture, a shutter, and the like, and with an imaging device such as a complementary metal-oxide semiconductor (CMOS) image sensor or a charge coupled device (CCD) image sensor or the like. The camera 42 images the surroundings of the user 20 (for example, an imaging range defined by an angle of view equivalent to the width of visual field of an ordinary healthy subject).

The communication I/F 44 is connected to the network 54. The communication I/F 44 and the communication I/F 26 perform the role of exchanging various information between the processor 46 and the processor 28 over the network 54. The exchange of various information between the processor 46 and the processor 28 is performed in a secure state using the communication I/F 44 and the communication I/F 26.

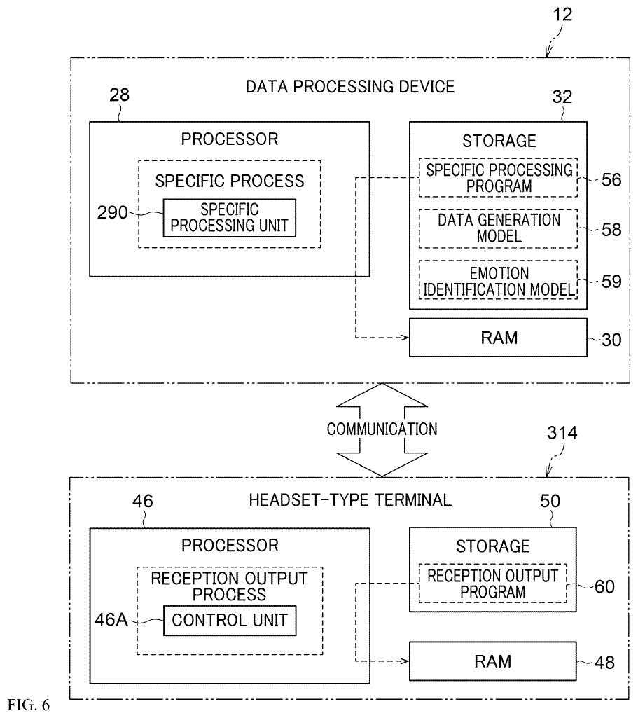

FIG. 6 illustrates an example of relevant functions of the data processing device 12 and the headset-type terminal 314. As illustrated in FIG. 6, specific processing is performed by the processor 28 in the data processing device 12. A specific processing program 56 is stored in the storage 32.

The specific processing program 56 is an example of a “program” according to technology disclosed herein. The processor 28 reads the specific processing program 56 from the storage 32, and in the RAM 30 executes the read specific processing program 56. The specific processing is implemented by the processor 28 operating as the specific processing unit 290 according to the specific processing program 56 executed in the RAM 30.

The data generation model 58 and the emotion identification model 59 are stored in the storage 32. The data generation model 58 and the emotion identification model 59 are employed by the specific processing unit 290.

Reception and output processing is performed by the processor 46 in the headset-type terminal 314. A reception and output program 60 is stored in the storage 50. The processor 46 reads the reception and output program 60 from the storage 50, and in the RAM 48 executes the read reception and output program 60. The reception and output processing is implemented by the processor 46 operating as the control unit 46A according to the reception and output program 60 executed in the RAM 48.

Next, description follows regarding the specific processing by the specific processing unit 290 of the data processing device 12. The units of the system described below are implemented by the data processing device 12 and the headset-type terminal 314. In the following description the data processing device 12 is called a “server”, and the headset-type terminal 314 is called a “terminal”.

### Example 1

Explanation of flow will be omitted due to being similar to a flow of the specific processing in Example 1 as described in the first exemplary embodiment above.

### Application Example 1

Explanation of flow will be omitted due to being similar to a flow of the specific processing in Application Example 1 as described in the first exemplary embodiment above.

### Example 2

Explanation of flow will be omitted due to being similar to a flow of the specific processing in Example 2 as described in the first exemplary embodiment above.

### Application Example 2

Explanation of flow will be omitted due to being similar to a flow of the specific processing in Application Example 2 as described in the first exemplary embodiment above.

The specific processing unit 290 transmits a result of the specific processing to the headset-type terminal 314. In the headset-type terminal 314, the control unit 46A outputs the result of the specific processing to the speaker 240 and the display 343. The microphone 238 acquires audio representing user input in response to the specific processing result. The control unit 46A transmits audio data representing the user input as acquired by the microphone 238 to the data processing device 12. The specific processing unit 290 in the data processing device 12 acquires the audio data.

The data generation model 58 is a so-called generative artificial intelligence (AI). Examples of the data generation model 58 include generative Als such as ChatGPT (registered trademark) (Internet search <URL: https://openai.com/blog/chatgpt>) and the like. The data generation model 58 is obtained by performing deep learning with a neural network. The data generation model 58 is input with a prompt including an instruction, and is input with inference data such as audio data representing speech, text data representing text, image data representing images (for example, still image data or video data), and the like. The data generation model 58 takes the input inference data, performs inference according to the instruction indicated in the prompt, and outputs an inference result in one or more data format from out of audio data, text data, image data, or the like. The data generation model 58 includes, for example, a text generative AI, an image generative AI, a multimodal generative AI, or the like. Reference here to inference indicates, for example, analysis, classification, prediction, and/or abstraction etc. The specific processing unit 290 performs the specific processing referred to above while using the data generation model 58. The data generation model 58 may be a model fine-tuned so as to output an inference result from a prompt not including an instruction, and in such cases the data generation model 58 is able to output an inference result from the prompt not including an instruction. There are plural types of the data generation model 58 included in the data processing device 12 or the like, and the data generation models 58 include an AI other than a generative AI. An AI other than a generative AI is, for example, a linear regression, a logistic regression, a decision tree, a random forest, a support vector machine (SVM), a k-means clustering, a convolutional neural network (CNN), a recurrent neural network (RNN), a generative adversarial network (GAN), a naïve Bayes, or the like and is capable of performing various processing, however there is no limitation to such examples. The AI may be an AI agent. Moreover, when the processing of each of the units mentioned above is performed by an AI, this processing is partly or entirely performed by the AI, however there is no limitation to such examples. Moreover, processing executed by an AI including a generative AI may be switched to rule-based processing, and rule-based processing may be switched to processing executed by an AI including a generative AI.

Although the processing by the data processing system 10 described above is executed by the specific processing unit 290 of the data processing device 12 or by the control unit 46A of the headset-type terminal 314, the processing may be executed by a specific processing unit 290 of the data processing device 12 and a control unit 46A of the headset-type terminal 314. Moreover, the specific processing unit 290 of the data processing device 12 acquires and collects information needed for processing from the headset-type terminal 314 or from an external device or the like, and the headset-type terminal 314 acquires and collects information needed for processing from the data processing device 12 or from an external device or the like.

For example, the collection unit is implemented by the control unit 46A of the headset-type terminal 314 and/or by the specific processing unit 290 of the data processing device 12. For example, an acquisition unit acquires number-of-steps data using the camera 42 and/or the communication I/F 44 of the headset-type terminal 314, and the number-of-steps data is processed by the specific processing unit 290 of the data processing device 12. For example, an analysis unit implemented by the specific processing unit 290 of the data processing device 12 analyzes data from the collection unit and the acquisition unit. For example, a generation unit implemented by the specific processing unit 290 of the data processing device 12 generates a cooking menu using a generative AI. For example, a supply unit implemented by the speaker 240 and the display 343 of the headset-type terminal 314 and/or the specific processing unit 290 of the data processing device 12 supplies the generated cooking menu to the user.

Correspondence relationships of each unit to devices and control units are not limited to the examples described above, and various modifications thereof are possible.

The above exemplary embodiment gives an implementation example in which the specific processing is performed by the data processing device 12, however technology disclosed herein is not limited thereto, and the specific processing may be performed by the headset-type terminal 314.

### Fourth Exemplary Embodiment

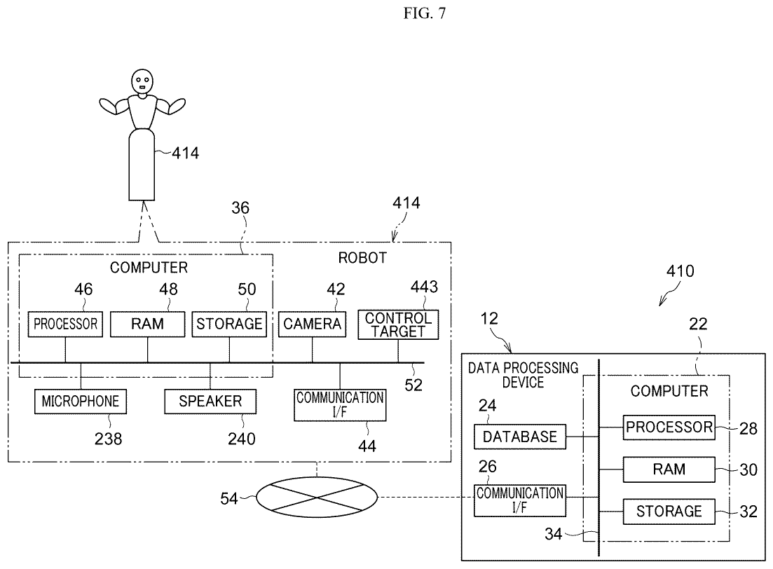

FIG. 7 illustrates an example of a configuration of a data processing system 410 according to a fourth exemplary embodiment

As illustrated in FIG. 7, the data processing system 410 includes a data processing device 12 and a robot 414. A server is an example of the data processing device 12.

The data processing device 12 includes a computer 22, a database 24, and a communication I/F 26. The computer 22 is an example of a “computer” according to technology disclosed herein. The computer 22 includes a processor 28, RAM 30, and storage 32. The processor 28, the RAM 30, and the storage 32 are connected to a bus 34. The database 24 and the communication I/F 26 are also connected to the bus 34. The communication I/F 26 is connected to a network 54. Examples of the network 54 include a Wide Area Network (WAN) and/or a local area network (LAN).

The robot 414 includes a computer 36, a microphone 238, a speaker 240, a camera 42, a communication I/F 44, and a control target 443. The computer 36 includes a processor 46, RAM 48, and storage 50. The processor 46, the RAM 48, and the storage 50 are connected to a bus 52. The microphone 238, the speaker 240, the camera 42, the control target 443, and the communication I/F 44 are also connected to the bus 52.

The microphone 238 receives an instruction or the like from a user 20 by receiving speech uttered by the user 20. The microphone 238 captures the speech uttered by the user 20, converts the captured speech into audio data, and outputs the audio data to the processor 46. The speaker 240 outputs audio under instruction from the processor 46.

The camera 42 is a compact digital camera installed with an optical system such as a lens, an aperture, a shutter, and the like, and with an imaging device such as a complementary metal-oxide semiconductor (CMOS) image sensor or a charge coupled device (CCD) image sensor or the like. The camera 42 images the surroundings of the robot 414 (for example, with an imaging range defined by an angle of view equivalent to the width of visual field of an ordinary healthy subject).

The communication I/F 44 is connected to the network 54. The communication I/F 44 and the communication I/F 26 perform the role of exchanging various information between the processor 46 and the processor 28 over the network 54. The exchange of various information between the processor 46 and the processor 28 is performed in a secure state using the communication I/F 44 and the communication I/F 26.

The control target 443 includes a display device, eye LEDs, and motors to drive arms, hands, feet, and the like. The posture and gesture of the robot 414 are controlled by controlling the motors of the arms, hands, feet, and the like. Part of an emotion of the robot 414 can be expressed by controlling these motors. Moreover, a facial expression of the robot 414 can be represented by controlling an illumination state of the eye LEDs of the robot 414.

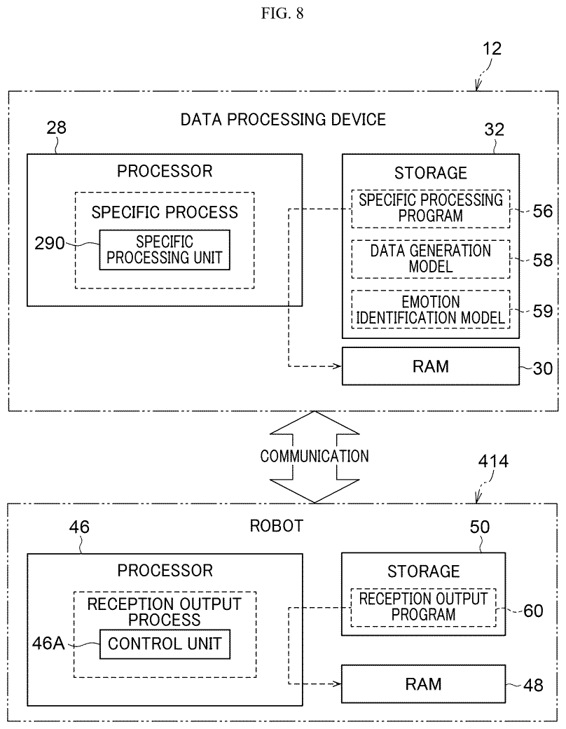

FIG. 8 illustrates an example of relevant functions of the data processing device 12 and the robot 414. As illustrated in FIG. 8, specific processing is performed by the processor 28 in the data processing device 12. A specific processing program 56 is stored in the storage 32.

The specific processing program 56 is an example of a “program” according to technology disclosed herein. The processor 28 reads the specific processing program 56 from the storage 32, and in the RAM 30 executes the read specific processing program 56. The specific processing is implemented by the processor 28 operating as the specific processing unit 290 according to the specific processing program 56 executed in the RAM 30.

The data generation model 58 and the emotion identification model 59 are stored in the storage 32. The data generation model 58 and the emotion identification model 59 are employed by the specific processing unit 290.

Reception and output processing is performed by the processor 46 in the robot 414. A reception and output program 60 is stored in the storage 50. The processor 46 reads the reception and output program 60 from the storage 50, and in the RAM 48 executes the read reception and output program 60. The reception and output processing is implemented by the processor 46 operating as the control unit 46A according to the reception and output program 60 executed in the RAM 48.

Next, description follows regarding the specific processing by the specific processing unit 290 of the data processing device 12. The units of the system described below are implemented by the data processing device 12 and the robot 414. In the following description the data processing device 12 is called a “server”, and the robot 414 is called a “terminal”.

### Example 1

Explanation of flow will be omitted due to being similar to a flow of the specific processing in Example 1 as described in the first exemplary embodiment above.

### Application Example 1

Explanation of flow will be omitted due to being similar to a flow of the specific processing in Application Example 1 as described in the first exemplary embodiment above.

### Example 2

Explanation of flow will be omitted due to being similar to a flow of the specific processing in Example 2 as described in the first exemplary embodiment above.

### Application Example 2

Explanation of flow will be omitted due to being similar to a flow of the specific processing in Application Example 2 as described in the first exemplary embodiment above.

The specific processing unit 290 transmits a result of the specific processing to the robot 414. In the robot 414, the control unit 46A outputs the result of the specific processing to the speaker 240 and the control target 443. The microphone 238 acquires audio representing user input in response to the specific processing result. The control unit 46A transmits audio data representing the user input as acquired by the microphone 238 to the data processing device 12. The specific processing unit 290 in the data processing device 12 acquires the audio data.

The data generation model 58 is a so-called generative artificial intelligence (AI). Examples of the data generation model 58 include generative Als such as ChatGPT (registered trademark) (Internet search <URL: https://openai.com/blog/chatgpt>) and the like. The data generation model 58 is obtained by performing deep learning with a neural network. The data generation model 58 is input with a prompt including an instruction, and is input with inference data such as audio data representing speech, text data representing text, image data representing images (for example, still image data or video data), and the like. The data generation model 58 takes the input inference data, performs inference according to the instruction indicated in the prompt, and outputs an inference result in one or more data format from out of audio data, text data, image data, or the like. The data generation model 58 includes, for example, a text generative AI, an image generative AI, a multimodal generative AI, or the like. Reference here to inference indicates, for example, analysis, classification, prediction, and/or abstraction etc. The specific processing unit 290 performs the specific processing referred to above while using the data generation model 58. The data generation model 58 may be a model fine-tuned so as to output an inference result from a prompt not including an instruction, and in such cases the data generation model 58 is able to output an inference result from the prompt not including an instruction. There are plural types of the data generation model 58 included in the data processing device 12 or the like, and the data generation models 58 include an AI other than a generative AI. An AI other than a generative AI is, for example, a linear regression, a logistic regression, a decision tree, a random forest, a support vector machine (SVM), a k-means clustering, a convolutional neural network (CNN), a recurrent neural network (RNN), a generative adversarial network (GAN), a naïve Bayes, or the like and is capable of performing various processing, however there is no limitation to such examples. The AI may be an AI agent. Moreover, when the processing of each of the units mentioned above is performed by an AI, this processing is partly or entirely performed by the AI, however there is no limitation to such examples. Moreover, processing executed by an AI including a generative AI may be switched to rule-based processing, and rule-based processing may be switched to processing executed by an AI including a generative AI.

Although the processing by the data processing system 10 described above is executed by the specific processing unit 290 of the data processing device 12 or by the control unit 46A of the robot 414, the processing may be executed by a specific processing unit 290 of the data processing device 12 and a control unit 46A of the robot 414. Moreover, the specific processing unit 290 of the data processing device 12 acquires and collects information needed for processing from the robot 414 or from an external device or the like, and the robot 414 acquires and collects information needed for processing from the data processing device 12 or from an external device or the like.

For example, the collection unit is implemented by the control unit 46A of the robot 414 and/or by the specific processing unit 290 of the data processing device 12. For example, an acquisition unit acquires number-of-steps data using the camera 42 and/or the communication I/F 44 of the robot 414, and the number-of-steps data is processed by the specific processing unit 290 of the data processing device 12. For example, an analysis unit implemented by the specific processing unit 290 of the data processing device 12 analyzes data from the collection unit and the acquisition unit. For example, a generation unit implemented by the specific processing unit 290 of the data processing device 12 generates a cooking menu using a generative AI. For example, a supply unit implemented by the speaker 240 and the control target 443 of the robot 414 and/or the specific processing unit 290 of the data processing device 12 supplies the generated cooking menu to the user. Correspondence relationships of each unit to devices and control units are not limited to the examples described above, and various modifications thereof are possible.

The above exemplary embodiment gives an implementation example in which the specific processing is performed by the data processing device 12, however technology disclosed herein is not limited thereto, and the specific processing may be performed by the robot 414.

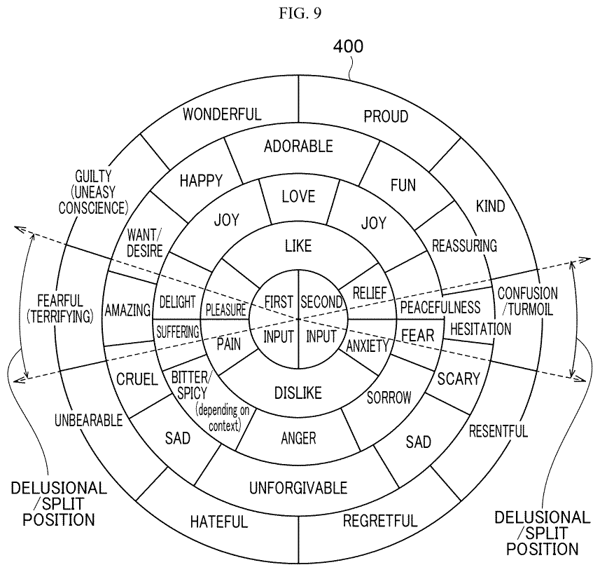

Note that the emotion identification model 59 serves as an emotion engine, and may decide the emotion of a user according to a specific mapping. Specifically, the emotion identification model 59 may decide the emotion of a user according to an emotion map (see FIG. 9) that is a specific mapping. Moreover, the emotion identification model 59 may also decide the emotion of the robot similarly, and the specific processing unit 290 may be configured so as to perform the specific processing using the emotion of the robot.

FIG. 9 is a diagram illustrating an emotion map 400 mapping plural emotions. In the emotion map 400, emotions are arranged in concentric circles that radiate out from the center. Primitive states of emotion are arranged nearer to the center of the concentric circles. Emotions expressing states and actions generated from states of mind are arranged further toward the outside of the concentric circles. Emotions are defined as including both affect and mental states. Emotions generated from reactions occurring in the brain are generally arranged at the left side of the concentric circles. Emotions induced by situational assessment are generally arranged at the right side of the concentric circles. Emotions generated from reactions occurring in the brain that are also emotions induced by situational assessment are generally arranged toward the top and toward the bottom of the concentric circles. Moreover, emotions of “euphoria” are arranged at the upper side of the concentric circles, and emotions of “dysphoria” are arranged at the lower side of the concentric circles. Plural emotions are accordingly mapped in this manner in the emotion map 400 based on a structure giving rise to emotions, and emotions that readily occur at the same time are mapped close to each other.

An example of such emotions is a distribution of emotions in the direction of 3 o'clock on the emotion map 400, generally around a boundary between relief and anxiety. Situational awareness dominates over internal sensations in the right half of the emotion map 400, with an impression of calm.

The inside of the emotion map 400 represents feelings, and the outside of the emotion map 400 represents actions, and so emotions further toward the outside of the emotion map 400 are more visible (are expressed by actions).

Human emotions are based on various balances, such as posture and blood sugar value balances, with a state of dysphoria being exhibited when these balances are far from ideal and a state of euphoria being exhibited when these balances are near to ideal. Even in a robot, a car, a motorbike, or the like, emotions can be thought of as being based on various balances such as orientation and remaining battery balances, with a state called dysphoria being exhibited when these balances are far from ideal and a state called euphoria being exhibited when these balances are near to ideal. An emotion map may, for example, be generated based on the emotion map of Dr. Mitsuyoshi (PhD Dissertation https://ci.nii.ac.jp/naid/500000375379: “Research on the phonetic recognition of feelings and a system for emotional physiological brain signal analysis”, Tokushima University). Emotions belonging to an area called “reaction” where feeling dominates are arranged in the left half of the emotion map. Moreover, emotions belonging to an area called “situation” where situational awareness dominates are arranged in the right half of the emotion map.

There are two types of emotion that facilitate leaning in an emotion map. One is an emotion in the vicinity of the center of negative “penitence” and “reflection” on the situational side. In other words, sometimes a negative “emotion” such as “I don't want to feel this way ever again” and “I don't want to be chided again” is experienced in a robot. Another is a positive emotion in the area of “desire” on the reaction side. In other words, there are times when a positive feeling such as “desire more” and “want to know more” is experienced.

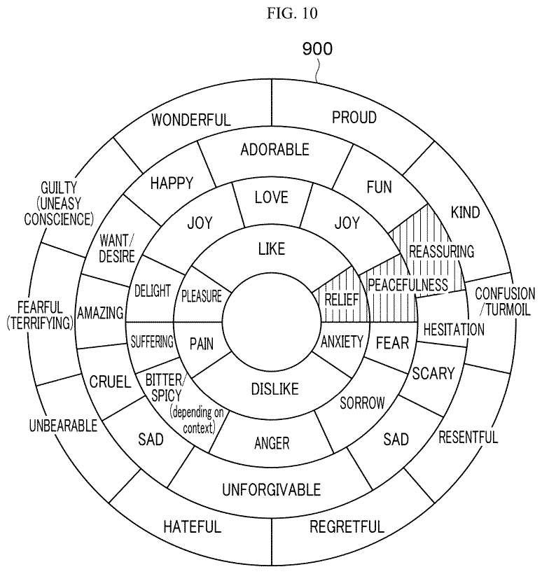

In the emotion identification model 59, user input is input to a pre-trained neural network, and emotion values indicating emotions shown on the emotion map 400 are acquired and the emotions of the user are decided. This neural network is pre-trained based on plural training data sets that each combine a user input with an emotion value indicating an emotion shown on the emotion map 400. The neural network is also trained such that emotions arranged close to each other have values that are close to each other, as in an emotion map 900 illustrated in FIG. 10. In FIG. 10 the plural emotions of “relief”, “peaceful”, and “reassured” are indicated as an example of close emotion values.

Although the system according to the present disclosure has been described mainly as functions of the data processing device 12, the system according to the present disclosure is not limited to being implemented in a server. The system according to the present disclosure may be implemented as a general information processing system. The present disclosure may, for example, be implemented by a software program operating on a personal computer, and may be implemented by an application operating on a smartphone or the like. The method according to the present disclosure may also be supplied to a user in the form of Software as a Service (Saas).

Although in the exemplary embodiments described above examples are given of embodiments in which the specific processing is performed by a single computer 22, technology disclosed herein is not limited thereto, and distributed processing may be performed for the specific processing, with the specific processing distributed across plural computers including the computer 22. For example, the data generation model 58 may be provided in a device external to the data processing device 12, such that data generation in response to input data is performed in the external device.

Although in the exemplary embodiments described above examples are described of embodiments in which the specific processing program 56 is stored in the storage 32, the technology disclosed herein is not limited thereto. For example, the specific processing program 56 may be stored on a portable, non-transitory, computer readable, storage medium, such as universal serial bus (USB) memory or the like. The specific processing program 56 stored on the non-transitory storage medium is then installed on the computer 22 of the data processing device 12. The processor 28 then executes the specific processing according to the specific processing program 56.

Moreover, the specific processing program 56 may be stored on a storage device, such as a server connected to the data processing device 12 over the network 54, with the specific processing program 56 then being downloaded in response to a request from the data processing device 12 and installed on the computer 22.

Note that there is no need to store the entire specific processing program 56 on the storage device, such as a server connected to the data processing device 12 over the network 54, or to store the entire specific processing program 56 on the storage 32, and part of the specific processing program 56 may be stored thereon.

Hardware resources for executing the specific processing may use various processors as listed below. Examples of processors include, for example, a CPU that is a general-purpose processor that functions as a hardware resource to execute the specific processing by executing software, namely a program. Moreover, the processor may, for example, be a dedicated electronic circuit that is a processor having a circuit configuration custom designed for executing the specific processing, such as a field-programmable gate array (FPGA), a programmable logic device (PLD), or an application specific integrated circuit (ASIC). Memory is inbuilt or connected to each of these processors, and the specific processing is executed by each of these processors using the memory.

The hardware resource that executes the specific processing may be configured from one of these various processors, or may be configured from a combination of two or more processors of the same or different type (for example, a combination of plural FPGAs, or a combination of a CPU and a FPGA). The hardware resource executing the specific processing may be a single processor.

Examples of configurations of a single processor include, firstly, a configuration of a single processor resulting from combining one or more CPU and software, in an embodiment in which this processor functions as the hardware resource for executing the specific processing. Secondly, as typified by a System-on-chip (SOC) or the like, there is also an embodiment that uses a processor realized by a single IC chip to function as an overall system including plural hardware resources for executing the specific processing. Adopting such an approach means that the specific processing is realized using one or more of the various processors described above as hardware resource.

Furthermore, more specifically, an electrical circuit that combines circuit elements such as semiconductor elements or the like may be employed as a hardware structure of these various processors. The specific processing is merely an example thereof. This means that obviously redundant steps may be omitted, new steps may be added, and the processing sequence may be swapped around within a range not departing from the spirit of the present disclosure.

The described content and drawing content illustrated above are a detailed description of parts according to the present disclosure, and are merely examples of the present disclosure. For example, description related to the above configuration, function, operation, and advantageous effects is a description related to examples of the configuration, function, operation, and advantageous effects of parts according to the present disclosure. This means that obviously redundant parts may be eliminated, new elements may be added, and switching around may be performed on the described content and drawing content illustrated above within a range not departing from the spirit of the present disclosure. Moreover, to avoid misunderstanding and to facilitate understanding of parts according to the present disclosure, description related to common knowledge in the art and the like not particularly needing description to enable implementation of the present disclosure is omitted in the described content and drawing content illustrated as described above.

All publications, patent applications and technical standards mentioned in the present specification are incorporated by reference in the present specification to the same extent as if each individual publication, patent application, or technical standard was specifically and individually indicated to be incorporated by reference.

Note that, regarding the above description, the following supplementary notes are further disclosed.

### Example 1

### (Supplementary 1)

A system comprising a processor,
wherein the processor is configured to
acquire time-series biological information from a plurality of detection devices, store the acquired biological information as time-series data in a storage device, perform preprocessing computation on the stored biological information and generate, for each predetermined time window, a sequence of feature values,
generate, on the basis of the sequence of feature values and a difference between the biological information and a reference state of the biological information, a prompt sentence in natural language that describes a trend of change in the biological information and a mental and physical state of a human,
input the prompt sentence and the sequence of feature values as input data to a generative AI model to cause the generative AI model to generate natural language output including an analysis result regarding the mental and physical state of the human and notification content to be presented to the human,
transmit, in accordance with the analysis result, a message including the notification content to an information processing apparatus operated by the human,
acquire evaluation information from the information processing apparatus, the evaluation information being given by the human in response to the message, and store a correspondence among the evaluation information, the prompt sentence, and the analysis result, and
generate, on the basis of the correspondence, learning data for updating the generative AI model or processing for generating the prompt sentence, and improve the generative AI model or the processing for generating the prompt sentence.

### (Supplementary 2)

The system according to supplementary 1,
wherein the processor is configured to
acquire, as the biological information, at least heart rate, blood pressure, skin electrical characteristics, and body temperature from the plurality of detection devices including sensors mounted on a body-worn device or a portable information terminal.

### (Supplementary 3)

The system according to supplementary 1,
wherein the processor is configured to
generate the prompt sentence so as to include an instruction to the generative AI model to
generate an action proposal for support, encouragement, or care for the human in consideration of an estimated mental and physical state of the human and a living situation of the human, and to cause the natural language output generated by the generative AI model to include the action proposal.

### Application Example 1

### (Supplementary 1)

A system comprising a processor,
wherein the processor is configured to
acquire, by using at least one sensor, measurement information relating to a biological state of a human subject,
transmit the measurement information to a server device via a terminal device, and store the measurement information in a storage unit of the server device,
analyze the stored measurement information in the server device by statistical processing or machine learning processing so as to determine presence or absence of an abnormal biological state and a duration of the abnormal biological state,
when the abnormal biological state satisfies a predetermined condition, generate a prompt sentence that instructs a generative AI model to generate content representing details of the abnormal biological state and recommended action guidance, and input the prompt sentence to the generative AI model,
generate, on the basis of an analysis result or action guidance obtained from the generative AI model, control information including warning information or explanatory information for a user, and transmit the control information to the terminal device so as to control a notification operation or a display operation of the terminal device,
when the abnormal biological state is determined to be a sustained abnormal state that continues for at least a predetermined time period, acquire, from the storage unit, contact information that has been registered in advance, generate a prompt sentence that instructs the generative AI model to generate a contact message relating to the sustained abnormal state, input the prompt sentence to the generative AI model, and transmit, via a communication network and on the basis of a message obtained from the generative AI model or a predefined template message, notification information to the contact information, and
dynamically generate prompt sentences for causing the generative AI model to generate at least one of analysis processing for the measurement information, a warning message for the user, and a notification message for the contact information, and update contents of the prompt sentences in accordance with changes in the biological state and analysis results.

### (Supplementary 2)

The system according to supplementary 1,
wherein the processor is configured to
cause the at least one sensor to acquire measurement information indicating a heart rate and a blood pressure, detect an abnormal state by comparing at least one of the heart rate and the blood pressure with a predetermined range, and change contents of a prompt sentence to be input to the generative AI model in accordance with the detected abnormal state.

### (Supplementary 3)

The system according to supplementary 1,
wherein the processor is configured to
generate a prompt sentence that instructs the generative AI model to provide information for reducing psychological burden of the user or supporting behavior of the user, input the prompt sentence to the generative AI model so as to generate a dialogue message relating to support, encouragement, or care for the user, and control presentation of information to the user via the terminal device on the basis of the dialogue message.

### Example 2

### (Supplementary 1)

A system comprising a processor,
wherein the processor is configured to
acquire biological information from a detection apparatus that measures biological information of a subject,
preprocess the acquired biological information, extract feature values based on time-series information included in the biological information, and estimate a mental and physical state of the subject based on the extracted feature values,
generate an analysis prompt sentence including the estimated mental and physical state and supplementary information related to the biological information, and input the analysis prompt sentence to a generative AI model to obtain one or more candidate response actions corresponding to the mental and physical state,
determine feedback content to be provided to the subject based on the one or more candidate response actions obtained from the generative AI model,
generate an operation prompt sentence including the determined feedback content, and input the operation prompt sentence to an information processing device so as to cause the information processing device to control an audio output apparatus or a display output apparatus, and
store the biological information, the estimated mental and physical state, and the feedback content in association with one another, and update content of at least one of the analysis prompt sentence and the operation prompt sentence based on stored information.

### (Supplementary 2)

The system according to Supplementary 1,
wherein the processor is configured to
handle the biological information as including at least heart rate information and blood pressure information, cause the detection apparatus to transmit the biological information to a terminal device via wireless communication, and cause the terminal device to aggregate the biological information and transmit the aggregated biological information to the information processing device via a communication network.

### (Supplementary 3)

The system according to Supplementary 1,
wherein the processor is configured to
include, in the analysis prompt sentence to be input to the generative AI model, an estimation result of the mental and physical state of the subject and a plurality of candidate assistance actions executable by the information processing device, obtain from the generative AI model a response specifying an action related to assistance, encouragement, or care for a human, and determine the feedback content based on the obtained response.

### Application Example 2

### (Supplementary 1)

A system comprising a processor,
wherein the processor is configured to
acquire biological information of a subject by using a sensor apparatus, the biological information including physiological state information, and to acquire at least one of behavior information and emotion information of the subject,
generate a first prompt sentence expressed in natural language, the first prompt sentence including at least an analysis condition, a state of the subject, and usage context derived from the biological information and the at least one of the behavior information and the emotion information, and input the first prompt sentence to a generative AI model to obtain, from the generative AI model, an analysis result or an action candidate relating to a physical and mental state or an emotional state of the subject,
generate a control prompt sentence defined in natural language, the control prompt sentence specifying an operation content of an information processing apparatus including at least one of an environment control apparatus and an information presentation apparatus, based on the analysis result or the action candidate, and input the control prompt sentence to the information processing apparatus to control an operation of the information processing apparatus, generate a support message or a proposal content using a natural language response obtained from the generative AI model, the natural language response being generated based on the analysis result or the action candidate and at least one of attribute information and history information of the subject, and output the support message or the proposal content to the subject or to an operator corresponding to the subject via a presentation apparatus including at least one of a display apparatus and an audio output apparatus, and
update at least one component of the first prompt sentence and the control prompt sentence based on biological information continuously acquired from the sensor apparatus and response information obtained from the subject, and iteratively optimize both input to the generative AI model and control of the operation of the information processing apparatus.

### (Supplementary 2)

The system according to supplementary 1,
wherein the processor is configured to
acquire, by using the sensor apparatus, a plurality of types of physiological state information including heart rate information and blood pressure information, associate the physiological state information with time information, calculate a feature quantity based on the physiological state information, and include the feature quantity in the first prompt sentence to be input to the generative AI model.

### (Supplementary 3)

The system according to supplementary 1,
wherein the processor is configured to
generate a second prompt sentence expressed in natural language, the second prompt sentence instructing the generative AI model to generate an action content related to support, encouragement, or consideration for a human, input the second prompt sentence to the generative AI model to obtain a natural language message corresponding to the physical and mental state or the emotional state of the subject, and present the natural language message to the subject or the operator via the information processing apparatus.

What is claimed is:

1. A system comprising:
circuitry configured to:
acquire biometric signal data from a sensor device, the biometric signal data including time-series physiological measurement values;
construct a first parameterized instruction sequence based on the biometric signal data, the first parameterized instruction sequence specifying an analysis task for a generative neural network model;
provide the first parameterized instruction sequence to the generative neural network model to cause the generative neural network model to generate analysis result data based on the biometric signal data; and
construct a second parameterized instruction sequence based on the analysis result data, and provide the second parameterized instruction sequence to an information processing apparatus to control an operation of the information processing apparatus based on the analysis result data.

2. The system according to claim 1, wherein the sensor device comprises at least one of a heart rate sensor, a blood pressure sensor, an electrodermal activity sensor, and an accelerometer, and wherein the biometric signal data includes at least heart rate values and blood pressure values sampled at predetermined intervals.

3. The system according to claim 2, wherein the circuitry is further configured to:
perform preprocessing on the biometric signal data including noise filtering, baseline correction, and normalization to generate preprocessed signal data, and incorporate the preprocessed signal data into the first parameterized instruction sequence.

4. The system according to claim 3, wherein the circuitry is further configured to:
compute derived feature values from the preprocessed signal data including heart rate variability metrics, mean arterial pressure values, and activity level indicators, and include the derived feature values in the first parameterized instruction sequence as structured context data.

5. The system according to claim 4, wherein the analysis result data includes at least one of a physiological state classification, an anomaly detection result, a stress level estimation, and a fatigue level estimation generated by the generative neural network model.

6. The system according to claim 5, wherein the circuitry is further configured to:
compare the physiological state classification against threshold conditions stored in a storage medium, and select a control action from a plurality of predefined control actions based on the comparison result, the control action specifying at least one of an environmental parameter adjustment, an alert generation, and an operational mode change for the information processing apparatus.

7. The system according to claim 6, wherein the second parameterized instruction sequence includes the selected control action and contextual parameters derived from the analysis result data, and wherein the information processing apparatus executes the control action by adjusting at least one of a display parameter, an audio output parameter, and a notification scheduling parameter.

8. The system according to claim 1, wherein the circuitry is further configured to:
construct a third parameterized instruction sequence instructing the generative neural network model to generate adaptive response data based on the analysis result data, the adaptive response data including at least one of contextual guidance text data, behavioral recommendation data, and motivational message data.

9. The system according to claim 8, wherein the adaptive response data is generated by the generative neural network model in a tone and content adapted to the physiological state classification, such that response content is adjusted based on detected stress levels and fatigue levels.

10. The system according to claim 9, wherein the circuitry is further configured to:
transmit the adaptive response data to a terminal apparatus via a packet-switched network for presentation on a display interface or via an audio output device associated with the terminal apparatus.

11. The system according to claim 1, wherein the generative neural network model comprises a transformer-based architecture including an embedding layer, a plurality of self-attention layers, feed-forward layers, and normalization layers, and wherein the circuitry provides the parameterized instruction sequence as a token sequence to the transformer-based architecture together with decoding control parameters including a maximum output token count and a sampling temperature value.

12. The system according to claim 11, wherein the circuitry is further configured to:
acquire environmental sensor data including temperature values, humidity values, and ambient light values from one or more environmental sensors, and incorporate the environmental sensor data into the first parameterized instruction sequence to provide contextual environmental information to the generative neural network model.

13. The system according to claim 12, wherein the analysis result data further includes environmental suitability assessment data generated by the generative neural network model based on correlation analysis between the biometric signal data and the environmental sensor data.

14. The system according to claim 1, wherein the circuitry is further configured to:
store the biometric signal data and the analysis result data in a storage medium in association with timestamp values and entity identification data, and compute longitudinal trend data by analyzing accumulated biometric signal data over a plurality of sessions.

15. The system according to claim 14, wherein the circuitry is further configured to:
incorporate the longitudinal trend data into the first parameterized instruction sequence to cause the generative neural network model to generate analysis result data that accounts for long-term physiological patterns of the entity.

16. The system according to claim 1, wherein the circuitry is further configured to:
acquire image data from an imaging device associated with the terminal apparatus, extract facial feature data from the image data using a facial landmark detection algorithm, apply an emotion estimation model to the facial feature data to compute emotion classification data, and incorporate the emotion classification data into the first parameterized instruction sequence as supplementary context data.

17. The system according to claim 16, wherein the emotion estimation model comprises a multi-layer neural network having an input layer, one or more hidden layers with non-linear activation functions, and an output layer that computes probability distributions over a set of emotion category labels.

18. A system comprising:
circuitry configured to:
acquire biometric signal data from a sensor device including at least heart rate values and blood pressure values;
perform preprocessing on the biometric signal data including noise filtering and normalization, and compute derived feature values including heart rate variability metrics;
construct a first parameterized instruction sequence based on the preprocessed signal data and the derived feature values, and provide the first parameterized instruction sequence to a generative neural network model having a transformer-based architecture including a plurality of self-attention layers to generate analysis result data including a physiological state classification;
construct a second parameterized instruction sequence based on the analysis result data to control an operation of an information processing apparatus; and
construct a third parameterized instruction sequence to cause the generative neural network model to generate adaptive response data based on the analysis result data, and transmit the adaptive response data to a terminal apparatus via a packet-switched network.

19. The system according to claim 18, wherein the circuitry is further configured to:
store the biometric signal data and the analysis result data in a storage medium, compute longitudinal trend data from accumulated sessions, and incorporate the longitudinal trend data into subsequent parameterized instruction sequences to account for long-term physiological patterns.

20. A method performed by circuitry, the method comprising:
acquiring biometric signal data from a sensor device, the biometric signal data including time-series physiological measurement values;
constructing a first parameterized instruction sequence based on the biometric signal data, the first parameterized instruction sequence specifying an analysis task for a generative neural network model;
providing the first parameterized instruction sequence to the generative neural network model to cause the generative neural network model to generate analysis result data based on the biometric signal data; and
constructing a second parameterized instruction sequence based on the analysis result data, and providing the second parameterized instruction sequence to an information processing apparatus to control an operation of the information processing apparatus based on the analysis result data.
